(19) United States
(12) Patent Application Publication
(10) Pub. No.: US 2024/0400658 A1
(43) Pub. Date: Dec. 5, 2024

(54) ANTI-PERIOSTIN HUMANIZED MONOCLONAL ANTIBODY, AND PREPARATION METHOD THEREFOR AND USE THEREOF

(71) Applicant: SHENYANG EYE INDUSTRY TECHNOLOGY INSTITUTE LTD., Liaoning (CN)

(72) Inventors: Xuejiao LI, Liaoning (CN); Wei HE, Liaoning (CN); Shuo ZHANG, Liaoning (CN); Hang ZHAO, Liaoning (CN); Ling FENG, Liaoning (CN); Hui LIU, Liaoning (CN); Hongli ZHAO, Liaoning (CN); Xiangdong HE, Liaoning (CN)

(21) Appl. No.: 18/691,316

(22) PCT Filed: Sep. 9, 2022
(86) PCT No.: PCT/CN2022/118162

§ 371 Date: Mar. 12, 2024

(30) Foreign Application Priority Data
Sep. 13, 2021 (CN) 202111068177.1

(51) Int. Cl. C07K 16/18; A61K 39/00; G01N 33/577

(52) U.S. Cl. CPC C07K 16/18; G01N 33/577; A61K 2039/505; C07K 2317/24; C07K 2317/73

(57) ABSTRACT

Provided in the present application are an anti-periostin humanized monoclonal antibody, and a preparation method therefor and the use thereof. The anti-periostin humanized monoclonal antibody has a relatively high expression level, and also has a better binding specificity and affinity to an antigen.

### CROSS REFERENCE TO RELATED APPLICATIONS

This application is a U.S. National Phase application under 35 U.S.C. § 371 of PCT Application No. PCT/CN2022/118162 filed Sep. 9, 2022, which claims the priority of Chinese Patent Application No. 202111068177.1, filed with the China National Intellectual Property Administration on Sep. 13, 2021, and titled with “anti-periostin humanized monoclonal antibody, and preparation method therefor and use thereof”, the disclosure of which is hereby incorporated by reference in its entirety.

### INCORPORATION OF SEQUENCE LISTING

The instant application contains a Sequence Listing which has been submitted in XML format and is hereby incorporated by reference in its entirety. Said XML copy, created on Mar. 12, 2024, is named UNITP0025US_Corrected_Sequence_Listing and is 53,972 bytes in size.

### FIELD

The present invention relates to the technical field of antibody drugs, and particularly relates to a humanized anti-periostin monoclonal antibody, and preparation method therefor and use thereof.

### BACKGROUND

Currently, vitreoretinal diseases such as proliferative diabetic retinopathy (PDR), proliferative vitreoretinopathy (PVR) and age-related macular degeneration (AMD) are the leading cause of vision impairment and vision loss in the corresponding population, and are also a medical problem worldwide. Therefore, it is necessary to find a novel biological label and therapeutic target for vitreoretinal diseases, which is essential for early detection, diagnosis, and treatment of these diseases.

Periostin is a cell adhesion protein found in mouse embryo osteoblast precursor cells in 1993 and named osteoblast-specific factor-2 (OSF-2). Due to its specifical expression in the periodontium and periosteum of adult mice, it is renamed periostin. Periostin is a unique extracellular matrix protein, which is involved in the immune-inflammatory responses corresponding to various tissues and organs of the human body, such as cardiac remodelling after myocardial infarction, myelofibrosis, regeneration and repair of periodontal tissues, cutaneous wound healing, tumor cell metastasis and renal injury, and induces chronic allergic diseases. In recent years, the research and production of periostin-targeted drugs has become a hotspot, and a large number of marketed drugs have achieved impressive success in tumor therapy.

The main roles of periostin in the eye include promoting cell proliferation, differentiation, migration and adhesion, inducing fiber formation and promoting neovascularization. It has been proved that periostin is a key factor in various vitreoretinal diseases in more and more domestic and international studies. Periostin is expected as a new biological label for vitreoretinal diseases, which will provide a new therapeutic strategy. For example, in many fields of ophthalmology, the periostin antibody has been reported to play a key role in the treatment of intraocular neovascular proliferation and macular oedema, etc., caused by age-related macular degeneration and diabetic retinopathy.

However, all existing periostin antibodies are murine monoclonal antibodies. As the murine antibodies are heterologous proteins, they may cause immune rejection against the heterologous proteins and induce the production of human anti-mouse antibodies in the human body, which shortens the half-life of the antibodies in vivo and impairs the therapeutic efficacy. Therefore, it is desirable to conduct antibody humanization on periostin antibody while maintaining a high affinity for specific antigenic epitopes to reduce the immunogenicity of the heterologous antibody and improve the specificity and affinity of binding to the antigen, leading to better therapeutic effects.

### SUMMARY

In view of this, the present invention aims to provide a humanized anti-periostin monoclonal antibody, preparation method and use thereof.

A humanized anti-periostin monoclonal antibody provided by the present invention comprises a heavy chain variable region and a light chain variable region, wherein the heavy chain variable region comprises an amino acid sequence set forth in SEQ ID NO: 1, and the light chain variable region comprises an amino acid sequence set forth in SEQ ID NO: 2.

The humanized anti-periostin monoclonal antibody provided by the present invention comprises a heavy chain constant region and a light chain constant region, wherein the heavy chain constant region is a heavy chain constant region of human IgG1, and the light chain constant region is a light chain constant region of kappa chain.

In some specific embodiments, the humanized anti-periostin monoclonal antibody comprises a heavy chain and a light chain, wherein the amino acid sequence of the heavy chain is set forth in SEQ ID NO: 3, and the amino acid sequence of the light chain is set forth in SEQ ID NO: 4.

In the present invention, antibody humanization is performed on a murine anti-periostin monoclonal antibody, and a eukaryotic expression vector is successfully constructed and transfected into mammalian CHO cells for expression and secretion. The biological functions such as affinity and binding activity are evaluated in vitro by SDS-PAGE, ELISA, WB, etc., which provides a theoretical basis for the large-scale expression of the humanized anti-periostin monoclonal antibody in CHO cells.

The anti-periostin humanized monoclonal antibody provided by the present invention comprises the above-mentioned heavy chain and light chain, which functions more safely and effectively than chimeric antibodies or other murine periostin monoclonal antibodies produced in the art. The humanized antibodies have been tested and shown to have good specificity against human periostin. It is confirmed that the periostin antibody plays a key role in the treatment of diseases such as intraocular neovascular proliferation and macular edema which are caused by age-related macular degeneration and diabetic retinopathy in many fields of ophthalmology, so the antibody provided by the present invention has the potential to treat tissue fibrosis.

The present invention further provides a nucleic acid encoding the anti-periostin humanized monoclonal antibody. Specifically, the present invention provides a nucleic acid encoding the heavy chain of said monoclonal antibody, a nucleic acid encoding the light chain of said monoclonal antibody, a nucleic acid encoding the heavy chain variable region of said monoclonal antibody, and/or a nucleic acid encoding the light chain variable region of said monoclonal antibody. In some embodiments, the nucleic acid encoding heavy chain of said antibody is set forth in SEQ ID NO: 5, and the nucleic acid encoding the light chain of said antibody is set forth in SEQ ID NO: 6. In order to facilitate protein expression, the 5′ end of the coding nucleic acid provided by the present invention further comprises a linker. Thus, in some specific embodiments, the nucleic acid encoding the heavy chain of the humanized anti-periostin monoclonal antibody is set forth in SEQ ID NO: 7; and the nucleic acid encoding the light chain of the humanized anti-periostin monoclonal antibody is set forth in SEQ ID NO: 8.

The present invention further provides an expression vector which comprises the nucleic acid encoding said monoclonal antibody. In the present invention, the expression vector comprises a backbone vector. In some embodiments, the backbone vector is pCDNA3.4.

The present invention further provides a host cell transformed or transfected with the expression vector. In the present invention, the host cell is a mammalian cell, and in some specific embodiments, the host cell is a CHO-K1 cell.

A method for producing the humanized anti-periostin monoclonal antibody of the present invention comprises culturing the host cell of the present invention and inducing the expression of the humanized anti-periostin monoclonal antibody.

The present invention further provides use of the humanized anti-periostin monoclonal antibody in the manufacture of a drug or a detection reagent, wherein the drug is a medicament for the treatment of tissue fibrosis and/or a malignant tumor, and the detection reagent is for diagnosing tissue fibrosis and/or a malignant tumor. In some embodiments, the tissue fibrosis is retinal fibrosis.

The present invention further provides a drug, which comprises the humanized anti-periostin monoclonal antibody of the present invention.

The present invention further provides a kit, which comprises the humanized anti-periostin monoclonal antibody, a composition formed by combining this antibody with a chemical label or a biological label, and a conjugate formed by coupling the humanized anti-periostin monoclonal antibody with a solid medium or a semi-solid medium.

In the present invention, the chemical label is isotopes, immunotoxins and/or chemical drugs and the biological label is biotin, affinities or enzyme labels. The enzyme label is preferably horseradish peroxidase or alkaline phosphatase. The immunotoxin is preferably aflatoxin, diphtheria toxin, Pseudomonas aeruginosa exotoxin, ricin, abrin, mistletoe lectin, modeccin, PAP, saporin, gelonin, or luffin. The solid medium or non-solid medium in the conjugate is selected from the group consisting of colloidal gold, polystyrene plates and beads. This kit can be used for diagnosing tissue fibrosis and/or a malignant tumor. The method for diagnosing employs the immunological detection techniques such as ELISA, Western blot, immunofluorescence techniques and immunohistochemistry assays.

The present invention further provides a method for the treatment of tissue fibrosis and/or a malignant tumor, which comprises administering the drug of the present invention.

The present invention further provides a method for diagnosing tissue fibrosis and/or a malignant tumor, in which the diagnosis is carried out by using the detection reagent of the present invention.

The present invention provides a humanized anti-periostin monoclonal antibody, in which only the complementary determining regions of the variable region from the murine McAb is retained, which bind to the antigen, and CDR grafting antibodies are produced. The humanized anti-periostin monoclonal antibodies have less immunogenicity compared to chimeric antibodies. In the experiments of the present invention, the obtained antibody could be produced in higher level, shows better specificity and affinity for binding to antigen compared to other antibodies, and involves in human immune response more effectively.

### BRIEF DESCRIPTION OF DRAWINGS

FIG. 1 shows the results for amplification of VH and VL gene derived from murine hybridoma cells (45-2-G3-1-G7-B7) by PCR.

FIG. 2 shows the PCR results after cloning.

FIGS. 3-1 and 3-2 show plasmid profiles containing heavy and light chains, respectively.

FIG. 4 shows the results of the purification.

FIG. 5 shows ELISA results for 18 humanized antibodies.

FIG. 6 shows the detection of expression vector by electrophoresis.

FIG. 7 shows the detection results of linearized expression vector by electrophoresis.

FIG. 8 shows the results of protein expression and purification after screening.

FIG. 9 shows the results of protein expression and purification in cells obtained after screening.

FIG. 10 shows the identification results for humanized anti-periostin antibodies 5-G4, 5-G6 and 5-G9.

FIG. 11 shows the results of tube formation inhibition assay, wherein the Periostin antibody is a humanized monoclonal antibody and the control Periostin is a murine monoclonal antibody.

FIG. 12 shows the results of the cell migration assay.

FIG. 13 shows the relative cell migration rate compared to the blank control group.

FIG. 14 shows the results of cell invasion assay.

FIG. 15 shows the relative cell invasion rate compared to the blank control group.

FIG. 16 shows the results of the cell scratch assay.

FIG. 17 shows the relative migration distance (%) of cells.

FIG. 18 shows the expression of VEGFA, α-SMA, Col I, Col III and Fibronectin proteins in RPE cells in different groups detected by WB.

FIG. 19 shows the expression levels of VEGFA, α-SMA, Col I, Col III and Fibronectin proteins in RPE cells in different groups.

FIG. 20 shows the mRNA expression of αvβ3, αvβ5 integrins, VEGFR-2, FAK, PI3K, p-PI3K, AKT and p-AKT on the membrane of vascular endothelial cells in different groups.

FIG. 21 shows the Western blot results of the tested proteins in HUVEC cells in different groups.

FIG. 22 shows the expression levels of tested proteins in HUVEC cells in different groups.

FIG. 23 shows the expression of FAK protein in HUVEC cells detected by IF.

FIG. 24 shows the expression of VEGFR2 protein in HUVEC cells detected by IF.

FIG. 25 shows the expression of αvβ3 protein in HUVEC cells detected by IF.

FIG. 26 shows the expression of αvβ5 protein in HUVEC cells detected by IF.

### DETAILED DESCRIPTION

The present invention provides a humanized anti-periostin monoclonal antibody, preparation method and use thereof. Those skilled in the art can learn from the contents of the disclosure and appropriately adapt the process parameters to achieve the disclosure. It should be noted that all similar replacements and modifications are obvious to those skilled in the art, and they are deemed to be included in the present invention. The method and use of the present invention have been described through preferred embodiments, and those skilled apparently can make modifications or appropriate changes and combinations of the method and use herein without departing from the content, spirit and scope of the present invention to realize and apply the technology of the present invention.

Unless defined otherwise, all technical and scientific terms used herein have the same meaning as understood by those ordinarily skilled in the art. For definitions and terms in the art, professionals can in particular refer to Current Protocols in Molecular Biology (Ausubel). The abbreviations for amino acid residues are the standard 3-letter and/or 1-letter codes used in the art to refer to one of the 20 commonly used L-amino acids.

The term “antibody” refers to a protein consisting of one or more polypeptides that bind specifically to an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions. The term “monoclonal antibody” refers to a preparation of an antibody molecule consisting of a single molecule. The monoclonal antibody composition shows a single binding specificity and affinity for a specific epitope.

The “variable region” of a heavy or light chain of an antibody is the N-terminal mature region of the chain. Currently known antibody include kappa and lambda light chains, and the alpha, gamma (IgG1, IgG2, IgG3, IgG4), delta, epsilon and mu heavy chains or equivalents in other species. Full-length immunoglobulin “light chains” (of about 25 kDa or about 214 amino acids) comprise a variable region of about 110 amino acids at the NH2-terminus and a kappa or lambda constant region at the COOH-terminus. Full-length immunoglobulin “heavy chains” (of about 50 kDa or about 446 amino acids), similarly comprise a variable region (of about 116 amino acids) and one of the heavy chain constant regions, e.g., gamma (of about 330 amino acids).

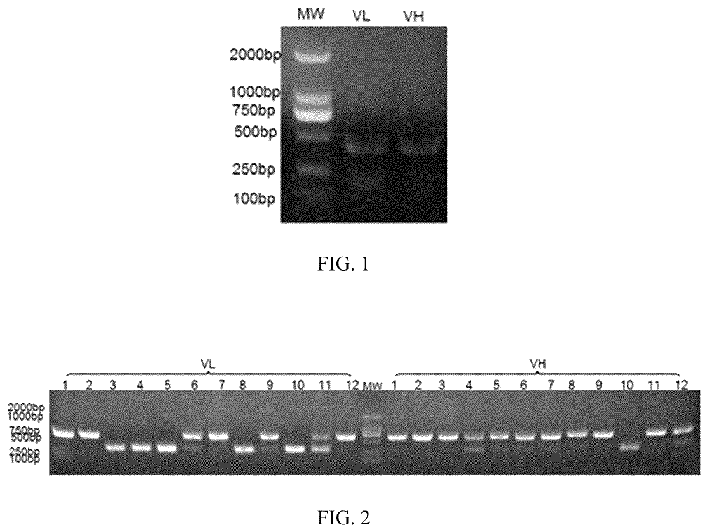

The terms “antibodies” include antibodies or immunoglobulins of any isotype and fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. Provided by the present invention is a humanized anti-periostin monoclonal antibody. Total RNA is extracted from the cultured hybridoma cell 45-2-G3-1-G7-B7. The cDNA is synthesized by reverse transcription using oligonucleotide primers, VH and VL fragments are amplified using IgG degenerate primers and kappa-specific primers, respectively, and the results are observed by gel electrophoresis (FIG. 1). Then, the PCR products are subcloned into standard vectors for clone picking and PCR verification (FIG. 2) and positive clones are sequenced. In order to construct the complete gene encoding the humanized anti-periostin monoclonal antibody and an eukaryotic expression vector containing this complete gene, the gene synthesis is used for replacing the gene encoding the framework region in the variable region of the murine anti-periostin monoclonal antibody with that of the human antibody, and recombining the gene fragment encoding the variable region of the humanized antibody into the vector containing the regulatory sequences and the gene encoding the human constant region. The expression vector is transfected into the CHO-K1 cells. CHO-K1 cell line that continuously and stably secretes the humanized antibody is obtained by screening with the GS system.

The amino acid sequence of the heavy chain variable region of the humanized anti-periostin monoclonal antibody of the present invention is

(SEQ ID NO: 1)
EVQLVQSGAEVKKPGESLKISCKASGYSFTDYFMNWVRQMPGKGLEWIGR
INPYSGDTLYNQRLQGQVTLSADKSISTAYLQLSSLKASDTAMYYCGRSG
VSGLDYWGQGTLVTVSS.

The amino acid sequence of the light chain variable region of the humanized anti-periostin monoclonal antibody of the present invention is

(SEQ ID NO: 2)
DIQLTQSPSSLSASVGDRVTITCSASSSASYMHWYQQKPGKAPKNWIYDT
SKLASGVPSRFSGSGSGTDYTLTISSLQPEDAATYYCQQWSSNPPTFGGG
TKVEIK.

The amino acid sequence of the heavy chain constant region of the humanized anti-periostin monoclonal antibody of the present invention is

(SEQ ID NO: 9)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.

The amino acid sequence of the light chain constant variable region of the humanized anti-periostin monoclonal antibody of the present invention is

(SEQ ID NO: 10)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The amino acid sequence of the heavy chain of the humanized anti-periostin monoclonal antibody of the present invention is

(SEQ ID NO: 3)
MKHLWFFLLLVAAPRWVLSEVQLVQSGAEVKKPGESLKISCKASGYSFTD
YFMNWVRQMPGKGLEWIGRINPYSGDTLYNQRLQGQVTLSADKSISTAYL
QLSSLKASDTAMYYCGRSGVSGLDYWGQGTLVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK.

The amino acid sequence of the light chain of the humanized anti-periostin monoclonal antibody of the present invention is

(SEQ ID NO: 4)
MVLQTQVFISLLLWISGAYGDIQLTQSPSSLSASVGDRVTITCSASSSAS
YMHWYQQKPGKAPKNWIYDTSKLASGVPSRFSGSGSGTDYTLTISSLQPE
DAATYYCQQWSSNPPTFGGGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC.

To facilitate the expression and purification of the antibodies, a linker is added to the N-terminus of the heavy-chain and light-chain fragment during vector construction and protein expression. This linker is removed during subsequent purification. In some embodiments, the amino acid sequence of the linker added at the N-terminus of the heavy chain of the humanized anti-periostin monoclonal antibody is MKHLWFFLLLVAAPRWVLS (SEQ ID NO: 11) and the amino acid sequence of the linker added at the N-terminus of the light chain of the humanized anti-periostin monoclonal antibody is MVLQTQVFISLLLWISGAYG (SEQ ID NO: 12).

Antibodies may be labeled for detection, for example, antibodies may be labeled and detected by using radioisotopes, enzymes that produce detectable substances, fluorescent proteins, biotin, and the like. Antibodies may also be bound to solid phase carriers including, but not limited to, polystyrene plates, beads, and the like.

The drug of the present invention comprises at least one pharmaceutical active ingredient, further comprising a pharmaceutically acceptable carrier. Preferably, the pharmaceutically acceptable carrier is water, buffered aqueous solution, isotonic salt solution such as PBS (phosphate buffer solution), glucose, mannitol, dextrose, lactose, starch, magnesium stearate, cellulose, magnesium carbonate, 0.3% glycerol, hyaluronic acid, ethanol and polyalkylene glycol such as polypropylene glycol, triglycerides and the like. The type of pharmaceutically acceptable carrier used is depends in particular on whether the composition of the present invention is to be administered orally, nasally, intradermally, subcutaneously, intramuscularly or intravenously. The composition according to the present invention may comprise wetting agents, emulsifiers or buffer substances as additives.

The reagents materials used in the present invention are all commercially available. The present invention is further described below in combination with examples.

### Example 1

### 1. Antibody Humanization Design

### (1) Verification of CDR Sequences

Total mRNA was extracted from murine hybridoma cells (45-2-G3-1-G7-B7, for the construction method, see “Chimeric anti-human periostin monoclonal antibody and use thereof”, application No. 202010788885.1, from Shenyang He's Eye Industry Group Co., Ltd./Shenyang Eye Industry Technology Research Institute Co., Ltd.) and used as a template for cDNA synthesis. The two DNA fragments encoding the heavy chain variable region and the light chain variable region were separated from the cDNA, cloned into the vector and sequenced. The following amino acid sequences were obtained:

>45-2-G3-1-G7-B7-VL1
(SEQ ID NO: 13)
DIVLTQSPAIMSASPGDKVTMTCSASSSASYMHWYQQKSGTSPKRWIYDT
SKLASGVPARFSGSGSGTSYSLTISSMEAEDAATYYCQQWSSNPPTFGGG
TKLEIK
>45-2-G3-1-G7-B7-VL2 and VL3
(SEQ ID NO: 14)
QIVLTQSPVIMSASPGDKVTMTCSASSSASYMHWYQQKSGTSPKRWIYDT
SKLASGVPARFSGSGSGTSYSLTISSMEAEDAATYYCQQWSSNPPTFGGG
TKLEIK
>45-2-G3-1-G7-B7-VH
(SEQ ID NO: 15)
EVQLQQSGPELVKPGASVRISCKASGYSFTDYFMNWVKQSHGRSLEWIGR
INPYSGDTLYNQRLKGKATLTVDKSSSTAHMELLSLTSEDSAVYYCGRSG
VSGLDYWGQGTSVTVSS

The amino acid sequences of the 3 CDRs in the light chain variable region are sequentially SSASY (SEQ ID NO: 16), DTS, and QQWSSNPPT (SEQ ID NO: 17).

The amino acid sequences of the 3 CDRs in the heavy chain variable region are sequentially GYSFTDYF (SEQ ID NO: 18), INPYSGDT (SEQ ID NO: 19) and GRSGVSGLDY (SEQ ID NO: 20).

### (2) Sequence Alignment and CDR Grafting of Murine Antibody

The sequences of the mouse antibody variable region were entered and searched in the IMGT mouse and human V genome databases using Igblast developed by NCBI. Sequence alignment was conducted to determine the human antibody variable region with the highest homology, and then the mouse antibody CDRs were grafted onto the human acceptor framework region to complete the CDR grafting.

### 2. Vector Construction and Expression for 18 Humanized Antibodies

### (1) Gene Synthesis

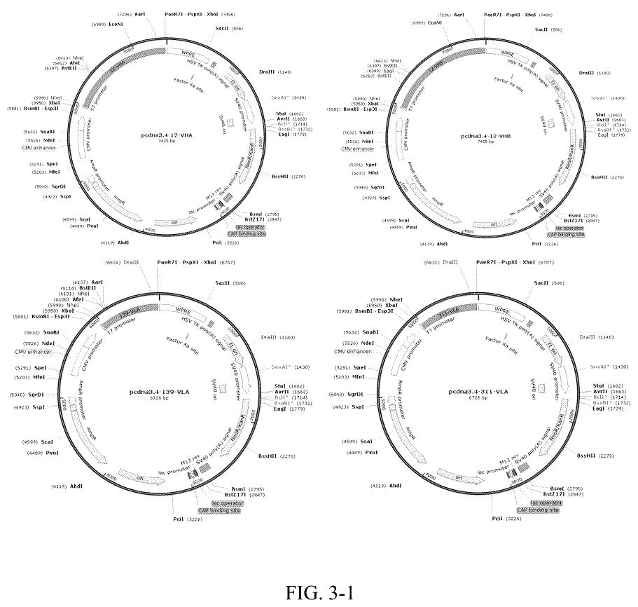

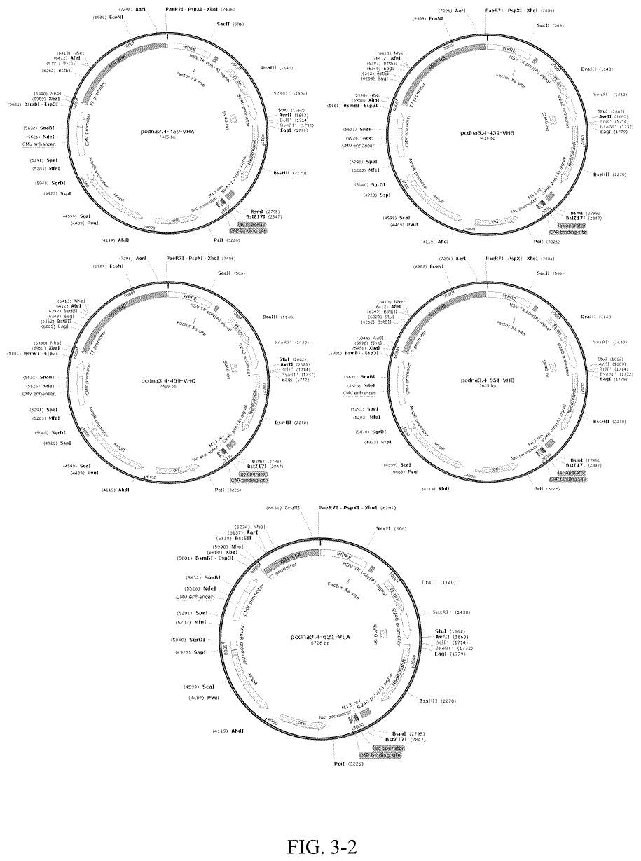

The DNA sequence of variable region was linked to that of human constant region and inserted human expression vector pcDNA3.4. The plasmid profiles are shown in FIG. 3-1 and FIG. 3-2. The nucleic acid sequences and amino acid sequences of the heavy chain and light chain of periostin antibody are as follows.

(2) Subcloning into Expression Vector

After gene synthesis, codon optimization was carried out for CHO system and the plasmid was subcloned into the PATX1 expression vector.

The nucleic acid sequences:

DNA sequence
>12-VHA-1423 bp
(SEQ ID NO: 21)
gaattcgccgccaccATGAAGCACCTGTGGTTTTTCCTGCTGCTGGTGGCCGCCCCCAGG
TGGGTTCTGTCCCAGGTGCAGCTGGTGCAGAGCGGCGCCGAGGTGAAGAAGCCTGG
CGCTTCCGTGAAGGTGTCCTGCAAGGCTTCTGGCTATAGCTTTACCGATTATTTCATGA
ACTGGGTGCGGCAGGCTCCTGGCCAGGGCTTGGAGTGGATCGGCAGGATCAACCCTT
ACTCCGGCGACACCCTGTATAACCAGAGGCTGAAGGGCCGGGCCACCCTGACCGTGG
ATAAGAGCATCAGCACCGCTTATATGGAGCTGTCCCGGCTGCGGTCCGACGACACCGC
TGTGTATTACTGCGGCAGGTCCGGCGTGTCCGGCCTGGATTACTGGGGCCAGGGCACC
CTGGTGACCGTGAGCAGCgctagcACCAAGGGACCTTCTGTGTTCCCTCTGGCTCCTTCT
TCTAAGTCCACTTCCGGTGGTACAGCAGCTCTGGGTTGTCTGGTGAAGGATTACTTCC
CAGAACCAGTGACTGTGTCCTGGAACTCCGGAGCTCTGACTTCTGGAGTGCATACTTT
CCCAGCAGTGCTGCAATCTAGCGGACTGTACTCTCTGTCTTCCGTGGTGACTGTGCCT
TCTTCTTCCCTGGGGACTCAAACTTACATCTGCAACGTGAACCACAAGCCCTCCAACA
CCAAGGTGGACAAGAAGGTGGAGCCAAAGAGCTGCGATAAGACCCACACCTGTCCA
CCTTGTCCAGCTCCAGAACTGCTGGGTGGGCCTTCTGTGTTTCTGTTCCCACCTAAGC
CAAAGGATACCCTGATGATCTCTAGGACCCCAGAAGTGACCTGTGTGGTCGTCGATGT
GTCTCATGAAGACCCTGAAGTGAAGTTCAACTGGTACGTGGACGGGGTGGAAGTGCA
TAACGCAAAGACCAAGCCCAGGGAAGAGCAATACAACTCCACCTACAGGGTGGTCTC
CGTCCTGACAGTCCTGCATCAGGATTGGCTGAACGGCAAGGAGTACAAGTGCAAGGT
CTCCAATAAAGCCCTGCCTGCCCCTATCGAGAAAACCATTAGCAAAGCCAAAGGCCA
GCCCAGGGAGCCCCAGGTCTATACACTGCCCCCCAGCAGGGAGGAGATGACAAAAAA
TCAGGTCAGCCTGACATGCCTGGTCAAAGGCTTTTATCCCAGCGACATTGCCGTCGAG
TGGGAGTCCAATGGCCAGCCCGAGAATAATTATAAAACAACACCCCCCGTCCTGGACA
GCGACGGCAGCTTTTTTCTGTATAGCAAACTGACAGTCGATAAAAGCAGGTGGCAGC
AGGGCAATGTCTTTTCCTGCAGCGTCATGCACGAGGCCCTGCACAATCACTATACTCA
GAAAAGCCTGAGCCTGTCCCCCGGGAAATGAGCGGCCGC
>12-VHB-1423 bp
(SEQ ID NO: 22)
gaattcgccgccaccATGAAGCACCTGTGGTTTTTCCTGCTGCTGGTGGCCGCTCCTCGGT
GGGTGCTGTCCCAGGTGCAGCTGGTGCAGAGCGGCGCCGAGGTGAAGAAGCCTGGC
GCTTCCGTGAAGGTGTCCTGTAAGGCCAGCGGCTACAGCTTCACCGACTACTTTATGA
ACTGGGTGAGGCAGGCTCCTGGCCAGGGCCTGGAGTGGATCGGCAGGATCAACCCCT
ATAGCGGCGACACCCTGTACAATCAGAAGCTGCAGGGCCGGGTGACCATGACCGTGG
ACAAGTCCATCAGCACCGCTTACATGGAGCTGTCCCGGCTGCGGAGCGACGATACCG
CTGTGTATTACTGCGGCCGGTCCGGCGTGAGCGGCTTGGATTATTGGGGCCAGGGCAC
CCTGGTGACCGTGAGCTCCgctagcACCAAGGGACCTTCTGTGTTCCCTCTGGCTCCTTC
TTCTAAGTCCACTTCCGGTGGTACAGCAGCTCTGGGTTGTCTGGTGAAGGATTACTTC
CCAGAACCAGTGACTGTGTCCTGGAACTCCGGAGCTCTGACTTCTGGAGTGCATACTT
TCCCAGCAGTGCTGCAATCTAGCGGACTGTACTCTCTGTCTTCCGTGGTGACTGTGCC
TTCTTCTTCCCTGGGGACTCAAACTTACATCTGCAACGTGAACCACAAGCCCTCCAAC
ACCAAGGTGGACAAGAAGGTGGAGCCAAAGAGCTGCGATAAGACCCACACCTGTCC
ACCTTGTCCAGCTCCAGAACTGCTGGGTGGGCCTTCTGTGTTTCTGTTCCCACCTAAG
CCAAAGGATACCCTGATGATCTCTAGGACCCCAGAAGTGACCTGTGTGGTCGTCGATG
TGTCTCATGAAGACCCTGAAGTGAAGTTCAACTGGTACGTGGACGGGGTGGAAGTGC
ATAACGCAAAGACCAAGCCCAGGGAAGAGCAATACAACTCCACCTACAGGGTGGTCT
CCGTCCTGACAGTCCTGCATCAGGATTGGCTGAACGGCAAGGAGTACAAGTGCAAGG
TCTCCAATAAAGCCCTGCCTGCCCCTATCGAGAAAACCATTAGCAAAGCCAAAGGCCA
GCCCAGGGAGCCCCAGGTCTATACACTGCCCCCCAGCAGGGAGGAGATGACAAAAAA
TCAGGTCAGCCTGACATGCCTGGTCAAAGGCTTTTATCCCAGCGACATTGCCGTCGAG
TGGGAGTCCAATGGCCAGCCCGAGAATAATTATAAAACAACACCCCCCGTCCTGGACA
GCGACGGCAGCTTTTTTCTGTATAGCAAACTGACAGTCGATAAAAGCAGGTGGCAGC
AGGGCAATGTCTTTTCCTGCAGCGTCATGCACGAGGCCCTGCACAATCACTATACTCA
GAAAAGCCTGAGCCTGTCCCCCGGGAAATGAGCGGCCGC
>459-VHA-1423 bp
(SEQ ID NO: 23)
gaattcgccgccaccATGAAGCACCTGTGGTTTTTCCTGCTGCTGGTGGCCGCCCCCCGGTGG
GTGCTGTCCCAGGTGCAGCTGCAGGAGAGCGGCCCTGGCCTGGTGAAGCCCAGCGA
GACCCTGAGCCTGACCTGCACCGCTTCCGGCTACAGCTTCACCGATTACTTCATGAAC
TGGGTGCGGCAGCCCCCTGGCAAGGGCTTGGAGTGGATCGGCAGGATCAACCCTTAC
AGCGGCGACACCCTGTATAATCAGCGGCTGAAGGGCAGGGTGACCCTGAGCGTGGAT
AAGAGCAAGAACCAGGCCAGCCTGAAGCTGAGCAGCGTGACCGCTGCCGATACCGC
CGTGTATTATTGTGGCCGGTCCGGCGTGAGCGGCCTGGATTACTGGGGCCAGGGCACC
CTGGTGACCGTGAGCAGCgctagcACCAAGGGACCTTCTGTGTTCCCTCTGGCTCCTTCT
TCTAAGTCCACTTCCGGTGGTACAGCAGCTCTGGGTTGTCTGGTGAAGGATTACTTCC
CAGAACCAGTGACTGTGTCCTGGAACTCCGGAGCTCTGACTTCTGGAGTGCATACTTT
CCCAGCAGTGCTGCAATCTAGCGGACTGTACTCTCTGTCTTCCGTGGTGACTGTGCCT
TCTTCTTCCCTGGGGACTCAAACTTACATCTGCAACGTGAACCACAAGCCCTCCAACA
CCAAGGTGGACAAGAAGGTGGAGCCAAAGAGCTGCGATAAGACCCACACCTGTCCA
CCTTGTCCAGCTCCAGAACTGCTGGGTGGGCCTTCTGTGTTTCTGTTCCCACCTAAGC
CAAAGGATACCCTGATGATCTCTAGGACCCCAGAAGTGACCTGTGTGGTCGTCGATGT
GTCTCATGAAGACCCTGAAGTGAAGTTCAACTGGTACGTGGACGGGGTGGAAGTGCA
TAACGCAAAGACCAAGCCCAGGGAAGAGCAATACAACTCCACCTACAGGGTGGTCTC
CGTCCTGACAGTCCTGCATCAGGATTGGCTGAACGGCAAGGAGTACAAGTGCAAGGT
CTCCAATAAAGCCCTGCCTGCCCCTATCGAGAAAACCATTAGCAAAGCCAAAGGCCA
GCCCAGGGAGCCCCAGGTCTATACACTGCCCCCCAGCAGGGAGGAGATGACAAAAAA
TCAGGTCAGCCTGACATGCCTGGTCAAAGGCTTTTATCCCAGCGACATTGCCGTCGAG
TGGGAGTCCAATGGCCAGCCCGAGAATAATTATAAAACAACACCCCCCGTCCTGGACA
GCGACGGCAGCTTTTTTCTGTATAGCAAACTGACAGTCGATAAAAGCAGGTGGCAGC
AGGGCAATGTCTTTTCCTGCAGCGTCATGCACGAGGCCCTGCACAATCACTATACTCA
GAAAAGCCTGAGCCTGTCCCCCGGGAAATGAGCGGCCGC
>459-VHB-1423 bp
(SEQ ID NO: 24)
gaattcgccgccaccATGAAGCACCTGTGGTTTTTCCTGCTGCTGGTGGCTGCTCCTCGGT
GGGTGCTGAGCCAGGTGCAGCTGCAGGAGAGCGGCCCCGGACTGGTGAAGCCTAGC
GAGACCCTGTCCCTGACCTGCACCGCCAGCGGCTATTCCTTTACCGATTATTTCATGAA
CTGGATCCGGCAGCCCCCTGGCAAGGGCCTGGAGTGGATCGGCCGGATCAACCCCTA
TTCCGGCGATACCCTGTACAACCAGCGGCTGAAGTCCAGGGTGACCCTGAGCGTGGA
CAAGTCCAAGAACCAGGCTTCCCTGAAGCTGTCCAGCGTGACCGCTGCTGATACCGC
TGTGTACTACTGCGGCCGGAGCGGCGTGTCCGGCTTGGATTATTGGGGCCAGGGCACC
CTGGTGACCGTGAGCAGCgctagcACCAAGGGACCTTCTGTGTTCCCTCTGGCTCCTTCT
TCTAAGTCCACTTCCGGTGGTACAGCAGCTCTGGGTTGTCTGGTGAAGGATTACTTCC
CAGAACCAGTGACTGTGTCCTGGAACTCCGGAGCTCTGACTTCTGGAGTGCATACTTT
CCCAGCAGTGCTGCAATCTAGCGGACTGTACTCTCTGTCTTCCGTGGTGACTGTGCCT
TCTTCTTCCCTGGGGACTCAAACTTACATCTGCAACGTGAACCACAAGCCCTCCAACA
CCAAGGTGGACAAGAAGGTGGAGCCAAAGAGCTGCGATAAGACCCACACCTGTCCA
CCTTGTCCAGCTCCAGAACTGCTGGGTGGGCCTTCTGTGTTTCTGTTCCCACCTAAGC
CAAAGGATACCCTGATGATCTCTAGGACCCCAGAAGTGACCTGTGTGGTCGTCGATGT
GTCTCATGAAGACCCTGAAGTGAAGTTCAACTGGTACGTGGACGGGGTGGAAGTGCA
TAACGCAAAGACCAAGCCCAGGGAAGAGCAATACAACTCCACCTACAGGGTGGTCTC
CGTCCTGACAGTCCTGCATCAGGATTGGCTGAACGGCAAGGAGTACAAGTGCAAGGT
CTCCAATAAAGCCCTGCCTGCCCCTATCGAGAAAACCATTAGCAAAGCCAAAGGCCA
GCCCAGGGAGCCCCAGGTCTATACACTGCCCCCCAGCAGGGAGGAGATGACAAAAAA
TCAGGTCAGCCTGACATGCCTGGTCAAAGGCTTTTATCCCAGCGACATTGCCGTCGAG
TGGGAGTCCAATGGCCAGCCCGAGAATAATTATAAAACAACACCCCCCGTCCTGGACA
GCGACGGCAGCTTTTTTCTGTATAGCAAACTGACAGTCGATAAAAGCAGGTGGCAGC
AGGGCAATGTCTTTTCCTGCAGCGTCATGCACGAGGCCCTGCACAATCACTATACTCA
GAAAAGCCTGAGCCTGTCCCCCGGGAAATGAGCGGCCGC
>459-VHC-1423 bp
(SEQ ID NO: 25)
gaattcgccgccaccATGAAGCACCTGTGGTTCTTTCTGCTGCTGGTGGCCGCCCCCAGG
TGGGTTCTGAGCCAGGTGCAGCTGCAGGAGAGCGGCCCCGGACTGGTGAAGCCTAG
CGAGACCCTGTCCCTGACCTGCACCGTGTCCGGCGGCTCCATCACCGATTATTTCATG
AACTGGATCAGGCAGCCCCCTGGCAAGGGCCTGGAGTGGATCGGCCGGATCAATCCC
TATAGCGGCGACACCCTGTACAATCAGCGGCTGAAGAGCAGGGTGACCCTGAGCGTG
GATAAGTCCAAGAATCAGGCCAGCCTGAAGCTGTCCTCCGTGACCGCCGCCGACACC
GCTGTGTACTACTGCGGCCGGTCCGGCGTGAGCGGCTTGGATTACTGGGGCCAGGGC
ACCCTGGTGACCGTGTCCAGCgctagcACCAAGGGACCTTCTGTGTTCCCTCTGGCTCCT
TCTTCTAAGTCCACTTCCGGTGGTACAGCAGCTCTGGGTTGTCTGGTGAAGGATTACT
TCCCAGAACCAGTGACTGTGTCCTGGAACTCCGGAGCTCTGACTTCTGGAGTGCATAC
TTTCCCAGCAGTGCTGCAATCTAGCGGACTGTACTCTCTGTCTTCCGTGGTGACTGTG
CCTTCTTCTTCCCTGGGGACTCAAACTTACATCTGCAACGTGAACCACAAGCCCTCCA
ACACCAAGGTGGACAAGAAGGTGGAGCCAAAGAGCTGCGATAAGACCCACACCTGT
CCACCTTGTCCAGCTCCAGAACTGCTGGGTGGGCCTTCTGTGTTTCTGTTCCCACCTA
AGCCAAAGGATACCCTGATGATCTCTAGGACCCCAGAAGTGACCTGTGTGGTCGTCGA
TGTGTCTCATGAAGACCCTGAAGTGAAGTTCAACTGGTACGTGGACGGGGTGGAAGT
GCATAACGCAAAGACCAAGCCCAGGGAAGAGCAATACAACTCCACCTACAGGGTGGT
CTCCGTCCTGACAGTCCTGCATCAGGATTGGCTGAACGGCAAGGAGTACAAGTGCAA
GGTCTCCAATAAAGCCCTGCCTGCCCCTATCGAGAAAACCATTAGCAAAGCCAAAGG
CCAGCCCAGGGAGCCCCAGGTCTATACACTGCCCCCCAGCAGGGAGGAGATGACAAA
AAATCAGGTCAGCCTGACATGCCTGGTCAAAGGCTTTTATCCCAGCGACATTGCCGTC
GAGTGGGAGTCCAATGGCCAGCCCGAGAATAATTATAAAACAACACCCCCCGTCCTG
GACAGCGACGGCAGCTTTTTTCTGTATAGCAAACTGACAGTCGATAAAAGCAGGTGG
CAGCAGGGCAATGTCTTTTCCTGCAGCGTCATGCACGAGGCCCTGCACAATCACTATA
CTCAGAAAAGCCTGAGCCTGTCCCCCGGGAAATGAGCGGCCGC
>551-VHB-1423 bp
(SEQ ID NO: 26)
gaattcgccgccaccATGAAGCACCTGTGGTTCTTCCTGCTGCTGGTGGCTGCTCCTAGGT
GGGTGCTGAGCGAGGTGCAGCTGGTGCAGAGCGGCGCCGAGGTGAAGAAGCCCGGC
GAGAGCCTGAAGATCTCCTGTAAGGCTTCCGGCTACTCCTTCACCGACTACTTTATGA
ATTGGGTGCGGCAGATGCCCGGCAAGGGCCTGGAGTGGATCGGCAGAATCAATCCTT
ACAGCGGCGACACCCTGTACAACCAGCGGCTGCAGGGCCAGGTGACCCTGTCCGCTG
ATAAGAGCATCTCCACCGCCTACCTGCAGCTGTCCTCCCTGAAGGCCTCCGACACCGC
CATGTACTACTGTGGCAGGAGCGGCGTGAGCGGCCTGGACTACTGGGGCCAGGGCAC
CCTGGTGACCGTGAGCAGCgctagcACCAAGGGACCTTCTGTGTTCCCTCTGGCTCCTTC
TTCTAAGTCCACTTCCGGTGGTACAGCAGCTCTGGGTTGTCTGGTGAAGGATTACTTC
CCAGAACCAGTGACTGTGTCCTGGAACTCCGGAGCTCTGACTTCTGGAGTGCATACTT
TCCCAGCAGTGCTGCAATCTAGCGGACTGTACTCTCTGTCTTCCGTGGTGACTGTGCC
TTCTTCTTCCCTGGGGACTCAAACTTACATCTGCAACGTGAACCACAAGCCCTCCAAC
ACCAAGGTGGACAAGAAGGTGGAGCCAAAGAGCTGCGATAAGACCCACACCTGTCC
ACCTTGTCCAGCTCCAGAACTGCTGGGTGGGCCTTCTGTGTTTCTGTTCCCACCTAAG
CCAAAGGATACCCTGATGATCTCTAGGACCCCAGAAGTGACCTGTGTGGTCGTCGATG
TGTCTCATGAAGACCCTGAAGTGAAGTTCAACTGGTACGTGGACGGGGTGGAAGTGC
ATAACGCAAAGACCAAGCCCAGGGAAGAGCAATACAACTCCACCTACAGGGTGGTCT
CCGTCCTGACAGTCCTGCATCAGGATTGGCTGAACGGCAAGGAGTACAAGTGCAAGG
TCTCCAATAAAGCCCTGCCTGCCCCTATCGAGAAAACCATTAGCAAAGCCAAAGGCCA
GCCCAGGGAGCCCCAGGTCTATACACTGCCCCCCAGCAGGGAGGAGATGACAAAAAA
TCAGGTCAGCCTGACATGCCTGGTCAAAGGCTTTTATCCCAGCGACATTGCCGTCGAG
TGGGAGTCCAATGGCCAGCCCGAGAATAATTATAAAACAACACCCCCCGTCCTGGACA
GCGACGGCAGCTTTTTTCTGTATAGCAAACTGACAGTCGATAAAAGCAGGTGGCAGC
AGGGCAATGTCTTTTCCTGCAGCGTCATGCACGAGGCCCTGCACAATCACTATACTCA
GAAAAGCCTGAGCCTGTCCCCCGGGAAATGAGCGGCCGC
>621-VLA-725 bp
(SEQ ID NO: 27)
gaattcgccgccaccATGGTGCTGCAGACCCAGGTGTTTATCTCCCTGCTGCTGTGGATCA
GCGGCGCCTATGGCGAGATCGTGCTGACCCAGTCCCCTGATTTCCAGAGCGTGACCCC
TAAGGAGAAGGTGACCATCACCTGCAGCGCCAGCAGCTCCGCCAGCTATATGCACTG
GTACCAGCAGAAGCCCGACCAGTCCCCTAAGCGGTGGATCTATGACACCAGCAAGCT
GGCTAGCGGCGTGCCTAGCAGGTTCTCCGGCAGCGGCAGCGGCACCGACTACACCCT
GACCATCAACTCCCTGGAGGCTGAGGACGCCGCCACCTATTACTGCCAGCAGTGGAG
CTCCAACCCCCCTACCTTTGGCGGCGGCACCAAGGTGGAGATCAAGcgtacgGTGGCTG
CACCTTCTGTGTTCATCTTCCCTCCATCTGATGAGCAGCTGAAGTCTGGAACCGCATCT
GTCGTCTGTCTGCTGAACAACTTTTACCCCAGGGAGGCTAAGGTCCAATGGAAGGTG
GACAACGCCCTGCAGTCTGGTAATAGCCAGGAAAGCGTGACCGAACAGGATTCCAAG
GACTCCACCTACTCCCTGTCCTCCACACTGACACTGAGCAAAGCCGACTATGAAAAG
CACAAAGTGTATGCCTGCGAGGTCACTCATCAGGGCCTGTCCAGCCCCGTGACTAAA
AGCTTTAATAGGGGGGAGTGCTGAGCGGCCGC
>311-VLA-725 bp
(SEQ ID NO: 28)
gaattcgccgccaccATGGTGCTGCAGACCCAGGTGTTTATCAGCCTGCTGCTGTGGATCA
GCGGCGCCTACGGCGAGATCGTGCTGACCCAGTCCCCCGCCACCCTGTCCCTGTCCCC
AGGAGAGAGGGCTACCCTGAGCTGCTCCGCCAGCTCCAGCGCCTCCTACATCCACTG
GTACCAGCAGAAGCCTGGCCAGGCCCCTCGGAGATGGATGTACGATACCTCCAAGCT
GGCCTCCGGCATCCCCGCCAGATTCAGCGGCAGCGGCAGCGGAACCGATTACACCCT
GACCATCAGCTCCCTGGAGCCTGAGGACGCCGCCGTGTACTACTGCCAGCAGTGGAG
CAGCAACCCTCCTACCTTCGGCGGCGGCACCAAGGTGGAGATCAAGcgtacgGTGGCTG
CACCTTCTGTGTTCATCTTCCCTCCATCTGATGAGCAGCTGAAGTCTGGAACCGCATCT
GTCGTCTGTCTGCTGAACAACTTTTACCCCAGGGAGGCTAAGGTCCAATGGAAGGTG
GACAACGCCCTGCAGTCTGGTAATAGCCAGGAAAGCGTGACCGAACAGGATTCCAAG
GACTCCACCTACTCCCTGTCCTCCACACTGACACTGAGCAAAGCCGACTATGAAAAG
CACAAAGTGTATGCCTGCGAGGTCACTCATCAGGGCCTGTCCAGCCCCGTGACTAAA
AGCTTTAATAGGGGGGAGTGCTGAGCGGCCGC
>139-VLA-725 bp
(SEQ ID NO: 29)
gaattcgccgccaccATGGTGCTGCAGACCCAGGTGTTTATCAGCCTGCTGCTGTGGATCA
GCGGCGCTTACGGCGACATCCAGCTGACCCAGTCCCCCTCCAGCCTGAGCGCTAGCG
TGGGCGACCGGGTGACCATCACCTGCTCCGCCTCCAGCTCCGCCAGCTACATGCACTG
GTACCAGCAGAAGCCCGGCAAGGCCCCCAAGAACTGGATCTATGATACCAGCAAGCT
GGCCAGCGGCGTGCCCAGCAGATTCAGCGGCAGCGGCTCCGGCACCGATTACACCCT
GACCATCAGCTCCCTGCAGCCCGAGGATGCTGCCACCTACTACTGCCAGCAGTGGTCC
TCCAACCCCCCTACCTTTGGCGGCGGCACCAAGGTGGAGATCAAGcgtacgGTGGCTGC
ACCTTCTGTGTTCATCTTCCCTCCATCTGATGAGCAGCTGAAGTCTGGAACCGCATCTG
TCGTCTGTCTGCTGAACAACTTTTACCCCAGGGAGGCTAAGGTCCAATGGAAGGTGG
ACAACGCCCTGCAGTCTGGTAATAGCCAGGAAAGCGTGACCGAACAGGATTCCAAGG
ACTCCACCTACTCCCTGTCCTCCACACTGACACTGAGCAAAGCCGACTATGAAAAGC
ACAAAGTGTATGCCTGCGAGGTCACTCATCAGGGCCTGTCCAGCCCCGTGACTAAAA
GCTTTAATAGGGGGGAGTGCTGAGCGGCCGC
>12-VHA
(SEQ ID NO: 30)
MKHLWFFLLLVAAPRWVLSQVQLVQSGAEVKKPGASVKVSCKASGYSFTDYFMN
WVRQAPGQGLEWIGRINPYSGDTLYNQRLKGRATLTVDKSISTAYMELSRLRSDDTAVYY
CGRSGVSGLDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK;
SIP:[1:19], Human IgG1 constant region: [137:466]
>12-VHB
(SEQ ID NO: 31)
MKHLWFFLLLVAAPRWVLSQVQLVQSGAEVKKPGASVKVSCKASGYSFTDYFMN
WVRQAPGQGLEWIGRINPYSGDTLYNQKLQGRVTMTVDKSISTAYMELSRLRSDDTAVY
YCGRSGVSGLDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK;
SIP:[1:19], Human IgG1 constant region: [137:466]
>459-VHA
(SEQ ID NO: 32)
MKHLWFFLLLVAAPRWVLSQVQLQESGPGLVKPSETLSLTCTASGYSFTDYFMNWV
RQPPGKGLEWIGRINPYSGDTLYNQRLKGRVTLSVDKSKNQASLKLSSVTAADTAVYYC
GRSGVSGLDYWGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK;
SIP:[1:19], Human IgG1 constant region: [137:466]
>459-VHB
(SEQ ID NO: 33)
MKHLWFFLLLVAAPRWVLSQVQLQESGPGLVKPSETLSLTCTASGYSFTDYFMNWIR
QPPGKGLEWIGRINPYSGDTLYNQRLKSRVTLSVDKSKNQASLKLSSVTAADTAVYYCGR
SGVSGLDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK;
SIP:[1:19], Human IgG1 constant region: [137:466]
>459-VHC
(SEQ ID NO: 34)
MKHLWFFLLLVAAPRWVLSQVQLQESGPGLVKPSETLSLTCTVSGGSITDYFMNWIR
QPPGKGLEWIGRINPYSGDTLYNQRLKSRVTLSVDKSKNQASLKLSSVTAADTAVYYCGR
SGVSGLDYWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK;
SIP:[1:19], Human IgG1 constant region: [137:466]
>551-VHB
(SEQ ID NO: 35)
MKHLWFFLLLVAAPRWVLSEVQLVQSGAEVKKPGESLKISCKASGYSFTDYFMNW
VRQMPGKGLEWIGRINPYSGDTLYNQRLQGQVTLSADKSISTAYLQLSSLKASDTAMYY
CGRSGVSGLDYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLSPGK;
SIP:[1:19], Human IgG1 constant region: [137:466]
>621-VLA
(SEQ ID NO: 36)
MVLQTQVFISLLLWISGAYGEIVLTQSPDFQSVTPKEKVTITCSASSSASYMHWYQQ
KPDQSPKRWIYDTSKLASGVPSRFSGSGSGTDYTLTINSLEAEDAATYYCQQWSSNPPTFG
GGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC;
SIP:[1:20], Human kappa constant region: [127:233]
>311-VLA
(SEQ ID NO: 37)
MVLQTQVFISLLLWISGAYGEIVLTQSPATLSLSPGERATLSCSASSSASYIHWYQQKP
GQAPRRWMYDTSKLASGIPARFSGSGSGTDYTLTISSLEPEDAAVYYCQQWSSNPPTFGG
GTKVEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC;
SIP:[1:20], Human kappa constant region: [127:233]
>139-VLA
(SEQ ID NO: 38)
MVLQTQVFISLLLWISGAYGDIQLTQSPSSLSASVGDRVTITCSASSSASYMHWYQQKPGK
APKNWIYDTSKLASGVPSRFSGSGSGTDYTLTISSLQPEDAATYYCQQWSSNPPTFGGGT
KVEIKRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC;
SIP:[1:20], Human kappa constant region: [127:233]

### (3) Transient Transfection

Endotoxin-free plasmid DNA was extracted. The CHO-K1 cells were co-transfected with the constructed vectors and then cultured. When the survival rate reduced below 50%, the culture supernatant was collected from a part of cells, antibodies were purified by using protein G and subjected to SDS-PAGE on gel. The activity of antibody was detected by ELISA using antigen protein.

### Transfection Method

<1> One day prior to transfection, 27×10⁶ cells were seeded in the complete culture medium (without antibiotics and anti-aggregation reagents) and reached logarithmic growth phase at the day of transfection.

<2> Preparation of a transfection mixture
a. DNA was diluted in a part of complete medium and mixed gently.
b. PEI transfection reagent was diluted in another part of complete medium, mixed gently, and incubated at room temperature for 5 minutes.
c. The diluted PEI was added to the diluted DNA in a total volume of 3 ml of the mixture, mixed gently, and incubated at room temperature for 20-30 minutes.

<3> Transfection was carried out as follows. 3 ml of the transfection mixture was added to 27 ml of cell suspension, mixed gently, and incubated at 37° C., 5% CO₂, 130 rpm until the cell survival rate reduces below 50%. Samples were collected.

Table 1 shows the details of the transfection.

TABLE 1

| | Combination of transfection | | |
|---|---|---|---|
| | (Name of Antibody) | HC | LC |
| 1 | 45-2-G3-1-G7-B7-12-VHA/621-VLA | 12-VHA | 621-VLA |
| 2 | 45-2-G3-1-G7-B7-12-VHA/311-VLA | | 311-VLA |
| 3 | 45-2-G3-1-G7-B7-12-VHA/139-VLA | | 139-VLA |
| 4 | 45-2-G3-1-G7-B7-12-VHB/621-VLA | 12-VHB | 621-VLA |
| 5 | 45-2-G3-1-G7-B7-12-VHB/311-VLA | | 311-VLA |
| 6 | 45-2-G3-1-G7-B7-12-VHB/139-VLA | | 139-VLA |
| 7 | 45-2-G3-1-G7-B7-459-VHA/621-VLA | 459-VHA | 621-VLA |
| 8 | 45-2-G3-1-G7-B7-459-VHA/311-VLA | | 311-VLA |
| 9 | 45-2-G3-1-G7-B7-459-VHA/139-VLA | | 139-VLA |
| 10 | 45-2-G3-1-G7-B7-459-VHB/621-VLA | 459-VHB | 621-VLA |
| 11 | 45-2-G3-1-G7-B7-459-VHB/311-VLA | | 311-VLA |
| 12 | 45-2-G3-1-G7-B7-459-VHB/139-VLA | | 139-VLA |
| 13 | 45-2-G3-1-G7-B7-459-VHC/621-VLA | | 621-VLA |
| 14 | 45-2-G3-1-G7-B7-459-VHC/311-VLA | 459-VHC | 311-VLA |
| 15 | 45-2-G3-1-G7-B7-459-VHC/139-VLA | | 139-VLA |
| 16 | 45-2-G3-1-G7-B7-551-VHB/621-VLA | | 621-VLA |
| 17 | 45-2-G3-1-G7-B7-551-VHB/311-VLA | 551-VHB | 311-VLA |
| 18 | 45-2-G3-1-G7-B7-551-VHB/139-VLA | | 139-VLA |

### (4) Purification

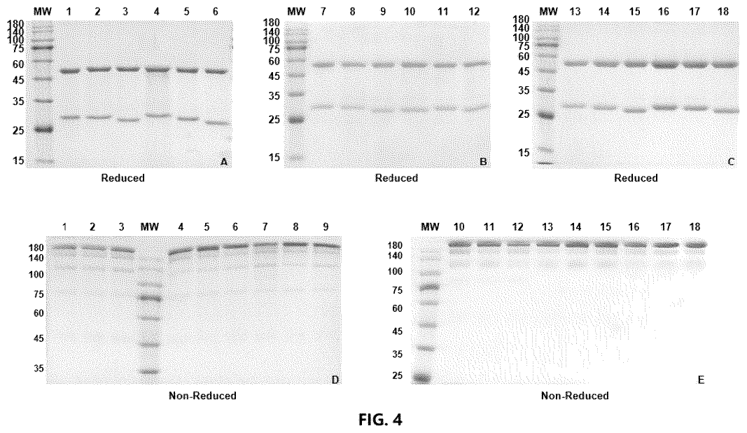

The supernatants were collected by centrifugation and loaded onto 18 Protein A purification columns (1 ml) respectively, and the packing was equilibrated with PBS with a pH of 7.5. The supernatant was mixed well with Protein A resin and incubated overnight and then purified. The PBS buffer with a pH of 7.0 was used for washing, 20 mM citric acid buffer with a pH of 2.7 was used for elution, and 1 M Tris HCl (neutralization buffer) was used for neutralization pH from 9.0 to 6.0. After purification, 20 μl of each of the IN, FT, washed samples and eluted samples were subjected to SDS-PAGE. The samples were collected based on the SDS-PAGE results and dialyzed overnight, and then the samples were pooled and subjected to the buffer exchange and concentration. The final results are shown in FIG. 4 and Table 2.

TABLE 2

| Number | Antibody | Concentration | Quantity | Number | Yield |
|---|---|---|---|---|---|
| 1 | 12-VHA/621-VLA | 1.05 mg/ml | 0.50 mg/vial | 1 vial | 0.50 mg |
| 2 | 12-VHA/311-VLA | 1.16 mg/ml | 0.50 mg/vial | 1 vial | 0.50 mg |
| 3 | 12-VHA/139-VLA | 1.31 mg/ml | 0.50 mg/vial | 1 vial | 0.50 mg |
| 4 | 12-VHB/621-VLA | 1.01 mg/ml | 0.50 mg/vial | 1 vial | 0.50 mg |
| 5 | 12-VHB/311-VLA | 1.00 mg/ml | 0.50 mg/vial | 1 vial | 0.50 mg |
| 6 | 12-VHB/139-VLA | 1.13 mg/ml | 0.50 mg/vial | 1 vial | 0.50 mg |
| 7 | 459-VHA/621-VLA | 0.96 mg/ml | 0.50 mg/vial | 1 vial | 0.50 mg |
| 8 | 459-VHA/311-VLA | 1.02 mg/ml | 0.50 mg/vial | 1 vial | 0.50 mg |
| 9 | 459-VHA/139-VLA | 1.07 mg/ml | 0.50 mg/vial | 1 vial | 0.50 mg |
| 10 | 459-VHB/621-VLA | 0.80 mg/ml | 0.50 mg/vial | 1 vial | 0.50 mg |
| 11 | 459-VHB/311-VLA | 1.13 mg/ml | 0.50 mg/vial | 1 vial | 0.50 mg |
| 12 | 459-VHB/139-VLA | 1.25 mg/ml | 0.50 mg/vial | 1 vial | 0.50 mg |
| 13 | 459-VHC/621-VLA | 0.69 mg/ml | 0.50 mg/vial | 1 vial | 0.50 mg |
| 14 | 459-VHC/311-VLA | 1.00 mg/ml | 0.50 mg/vial | 1 vial | 0.50 mg |
| 15 | 459-VHC/139-VLA | 1.16 mg/ml | 0.50 mg/vial | 1 vial | 0.50 mg |
| 16 | 551-VHB/621-VLA | 1.78 mg/ml | 0.50 mg/vial | 1 vial | 0.50 mg |
| 17 | 551-VHB/311-VLA | 1.71 mg/ml | 0.50 mg/vial | 1 vial | 0.50 mg |
| 18 | 551-VHB/139-VLA | 1.80 mg/ml | 0.50 mg/vial | 1 vial | 0.50 mg |

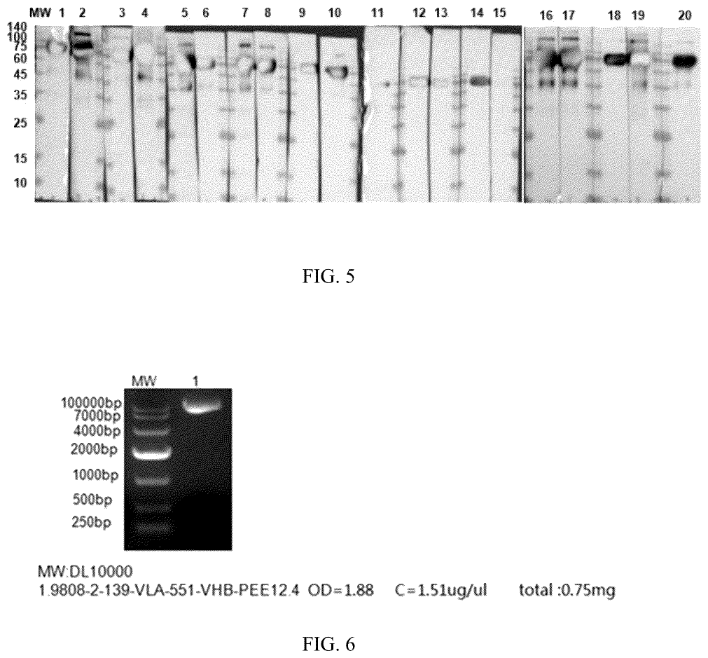

The specific binding was verified by Western Blot. The results are shown in Table 3 and FIG. 5, wherein 1-18 represent 18 humanized monoclonal antibodies, the 19009414P 11 (human IgG1) represents a chimeric periostin antibody and the 45-2-G3-1-G7-B7 represents a murine periostin monoclonal antibody.

TABLE 3

| Periostin-Mouse | Primary antibody | Secondary antibody | |
|---|---|---|---|
| 1 | 12-VHA/621-VLA | Goat Anti Human | Positive |
| 2 | 12-VHA/311-VLA | | Positive |
| 3 | 12-VHA/139-VLA | | Positive |
| 4 | 12-VHB/621-VLA | | positive |
| 5 | 12-VHB/311-VLA | | Positive |
| 6 | 12-VHB/139-VLA | | Positive |
| 7 | 459-VHA/621-VLA | | Positive |
| 8 | 459-VHA/311-VLA | | Positive |
| 9 | 459-VHA/139-VLA | | Positive |
| 10 | 459-VHB/621-VLA | | Positive |
| 11 | 459-VHB/311-VLA | | Positive |
| 12 | 459-VHB/139-VLA | | Positive |
| 13 | 459-VHC/621-VLA | | Positive |
| 14 | 459-VHC/311-VLA | | Positive |
| 15 | 459-VHC/139-VLA | | Positive |
| 16 | 551-VHB/621-VLA | | Positive |
| 17 | 551-VHB/311-VLA | | Positive |
| 18 | 551-VHB/139-VLA | | Positive |
| 19 | 19009414P 11 | | Positive |
| | (human IgG1) | | |
| 20 | 45-2-G3-1-G7-B7 | Goat Anti Mouse | Positive |

Arrangement of humanized antibodies on a plate for ELISA is shown in Table 4.

TABLE 4

| 1 | 2 | 3 | 4 | 5 | 6 | 7 | 8 |
|---|---|---|---|---|---|---|---|
| 12-VHA/ | 12-VHA/ | 12-VHA/ | 459-VHA/ | 459-VHA/ | 459-VHA/ | 551-VHB/ | 19009414P11 |
| 621-VLA | 621-VLA | 621-VLA | 139-VLA | 139-VLA | 139-VLA | 311-VLA | |
| 12-VHA/ | 12-VHA/ | 12-VHA/ | 459-VHB/ | 459-VHB/ | 459-VHB/ | 551-VHB/ | 19009414P11 |
| 311-VLA | 311-VLA | 311-VLA | 621-VLA | 621-VLA | 621-VLA | 311-VLA | |
| 12-VHA/ | 12-VHA/ | 12-VHA/ | 459-VHB/ | 459-VHB/ | 459-VHB/ | 551-VHB/ | 19009414P11 |
| 139-VLA | 139-VLA | 139-VLA | 311-VLA | 311-VLA | 311-VLA | 311-VLA | |
| 12-VHB/ | 12-VHB/ | 12-VHB/ | 459-VHB/ | 459-VHB/ | 459-VHB/ | 551-VHB/ | 45-2-G3-1- |
| 621-VLA | 621-VLA | 621-VLA | 139-VLA | 139-VLA | 139-VLA | 139-VLA | G7-B7 |
| 12-VHB/ | 12-VHB/ | 12-VHB/ | 459-VHC/ | 459-VHC/ | 459-VHC/ | 551-VHB/ | 45-2-G3-1- |
| 311-VLA | 311-VLA | 311-VLA | 621-VLA | 621-VLA | 621-VLA | 139-VLA | G7-B7 |
| 12-VHB/ | 12-VHB/ | 12-VHB/ | 459-VHC/ | 459-VHC/ | 459-VHC/ | 551-VHB/ | 45-2-G3-1- |
| 139-VLA | 139-VLA | 139-VLA | 311-VLA | 311-VLA | 311-VLA | 139-VLA | G7-B7 |
| 459-VHA/ | 459-VHA/ | 459-VHA/ | 459-VHC/ | 459-VHC/ | 459-VHC/ | Negative | Negative |
| 621-VLA | 621-VLA | 621-VLA | 139-VLA | 139-VLA | 139-VLA | control | control |
| 459-VHA/ | 459-VHA/ | 459-VHA/ | 551-VHB/ | 551-VHB/ | 551-VHB/ | Negative | Negative |
| 311-VLA | 311-VLA | 311-VLA | 621-VLA | 621-VLA | 621-VLA | control | control |

Results of ELISA assay are shown in Table 5.

TABLE 5

| 1 | 2 | 3 | 4 | 5 | 6 | 7 | 8 |
|---|---|---|---|---|---|---|---|
| 2.2676 | 2.1962 | 2.1325 | 1.0578 | 1.0122 | 1.0485 | 2.0413 | 1.7380 |
| 2.3149 | 2.2910 | 2.1548 | 1.9094 | 1.9964 | 1.9358 | 1.9902 | 1.6759 |
| 1.8726 | 1.9203 | 1.8613 | 2.0560 | 1.8604 | 1.8602 | 1.9714 | 1.6779 |
| 2.1021 | 1.9955 | 1.9839 | 0.4797 | 0.4511 | 0.4934 | 1.7166 | 0.7665 |
| 2.1828 | 2.1139 | 2.0769 | 1.1582 | 1.0934 | 1.0282 | 1.7625 | 0.7699 |
| 2.0264 | 2.1278 | 2.1131 | 0.6429 | 0.5133 | 0.5998 | 1.7652 | 0.6939 |
| 1.8504 | 1.9261 | 1.9378 | 0.0641 | 0.0639 | 0.0626 | 0.0619 | 0.0555 |
| 2.0901 | 2.2168 | 2.1762 | 2.0171 | 2.1129 | 2.0738 | 0.0554 | 0.0567 |

### 3. Construction of the Stable Transgenic Cell Line and Expression of the Humanized Antibody

### (1) Construction of Vector

From the results of transient transfection, it can be seen that the number 18th antibody 551-VHB/139-VLA has the highest expression and the binding ability meets the requirements. Therefore, considering the expression and the binding ability of antibodies, 551-VHB and 139-VLA were selected to use as the heavy and light chain of the humanized periostin antibody. The DNAs encoding 551-VHB and 139-VLA were subcloned into the pATX-GS2 expression vector respectively, and then co-transfected into CHO cells to express the antibody protein. The results of agarose gel electrophoresis are shown in FIG. 6.

### (2) Expression of the Humanized Periostin Antibody

The target fragment was subcloned into the expression vector pATX-GS2 after double enzyme digestion (EcoR I/Not I) and ligation, and then stably transfected into CHO cells. Stable cell lines were obtained through MSX pressure screening and limited dilution by ELISA. The stable cell line was incubated in a shaker incubator with 5% CO₂ at 37° C., for 7 days, and then the supernatant was collected, and the humanized antibodies in the supernatant were purified by affinity chromatography column packed with agarose gel-Protein A.

### <1> Linearization of Plasmid

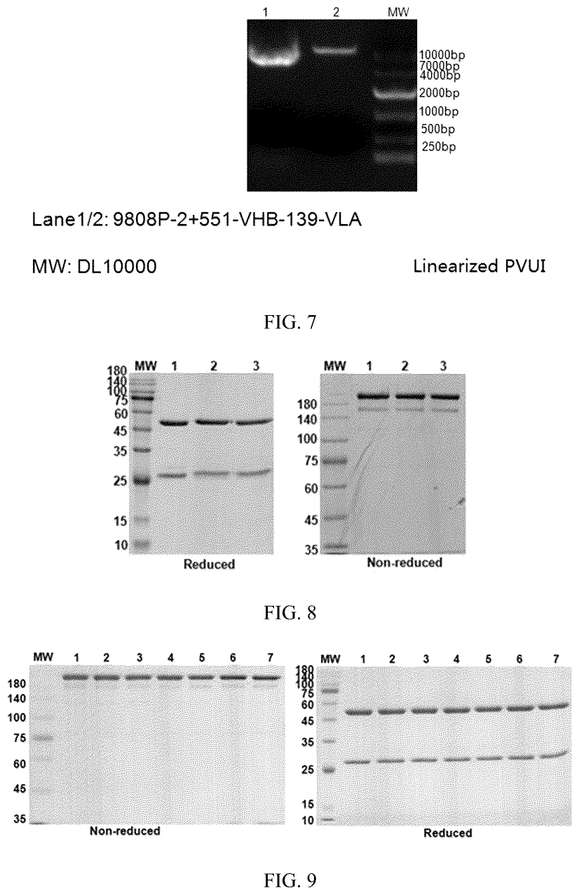

10 μl of extracted plasmid PATX-GS2-139-VLA-551-VHB (backup) was reserved in advance to verify the success of linearization. Enzymes and buffer were added to the plasmid for linearization sequentially and mixed well. The EP tube was inserted into the water-bath float, and putted into the water-bath at 37° C. to perform the enzyme digestion for about 1-2 hours. After enzymatic digestion, the plasmid was recovered as follows. 0.75 times isopropanol (based on the volume of the digestion system) was added to the digestion system and mixed well, and centrifuged at 4° C. for 30 minutes. The supernatant was discarded; 1 ml of 70% ethanol was added and centrifuged at 4° C. for 5 minutes. The concentration was measured by agarose gel electrophoresis (FIG. 7).

Enzyme digestion system is shown in Table 6.

TABLE 6

| | Volume |
|---|---|
| Reagents | (μl) |
| Buffer | 10 |
| Plasmid | all |
| Enzyme | 6-10 |

### <2> Transfection

CHO-K1 cells were transfected chemically with PVUI-linearized plasmid pATX-GS2-139-VLA-551-VHB by using FectoCHO™ Expression System Transfection Kit to obtain a stable transgenic cell pool expressing recombinant antibody.

### <3> Screening by Pressure

At 48 hours post-transfection, the transfected host cells were selected by using GS selection system (glutamine synthetase Gene Expression System). The cells were treated under selective pressure using 30 μM MSX. The natural resistance of the CHO-K1 cells to MSX was determined separately in 6-well plates by standard batch culture. The minimum killing concentration of MSX against non-transfected CHO-K1 cells was determined to be 20 μM. The transfected cells were treated with 30 μM MSX. The medium was replaced with fresh medium every 3-4 days until a stable cell line with resistance was generated. 3 stable cell pools were generated after 2 to 3 weeks under MSX selection.

Cells were cultured in 30 ml selective medium in a 125 ml shake flask under standard conditions (37° C., 5% CO₂, 130 rpm). For expression assay, when the cells were in good condition in the shake flask, a 30 ml fed-batch culture was carried out. Media supplement was added on D3, D5, D7 and D9 respectively and the glucose was monitored throughout. The cells were cultured consistently for consecutive 3 passages until the cell viability reduced below 50%, and the supernatants of the culture were collected, purified and analyzed. The purification results are shown in FIG. 8, where “reduced” represents the results of reduced SDS-PAGE and “non-reduced” represents the results of non-reduced SDS-PAGE.

Sample information is shown in Table 7.

TABLE 7

| Antibody | Concentration | Specification | Quantity | Yield |
|---|---|---|---|---|
| 18-stable 1 | 0.86 mg/ml | 1.75 ml/vial | 4 vials | 6.02 mg/30 ml |
| 18-stable 2 | 0.94 mg/ml | 1.80 ml/vial | 4 vials | 6.76 mg/30 ml |
| 18-stable 3 | 1.62 mg/ml | 1.80 ml/vial | 4 vials | 11.66 mg/30 ml |

### (4) Isolation of Monoclonal Antibodies by Limiting Dilution

The stable pool 3 (18-stable 3) polyclonal cells were selected and plated for limiting dilution under selective pressure. The cell suspension of the obtained stable transgenic pool was diluted to a very low density, allowing an average of 1 cell per well. A total of 7 monoclonal cell lines, 5-G4, 5-E6, 5-G6, 5-D7, 5-G9, 2-H3 and 2-C7, with high expression were selected after ELISA for subsequent expansion culture. The antibody concentrations is sequentially 1, 0.33, 0.11, 0.037, 0.012, 0.004, 0.0014 μg/ml (1:3-fold dilution gradient), plus a blank control for a total of 8 gradients. The mono-clones were observed under the microscope and the positive mono-clones were detected by ELSIA. The results of ELISA are shown Table 8.

TABLE 8

| | 5-E6 | | 2-C7 | | 5-G4 | | 5-G6 | | 5-G9 | |
|---|---|---|---|---|---|---|---|---|---|---|
| A | 3.396 | 3.3316 | 3.4232 | 3.5079 | 3.3039 | 3.4063 | 3.3018 | 3.4721 | 3.2732 | 3.2581 |
| B | 2.6476 | 2.546 | 2.9524 | 2.9827 | 2.5342 | 2.6667 | 2.6281 | 2.8172 | 2.5232 | 2.4339 |
| C | 2.0211 | 2.0248 | 2.4196 | 2.4409 | 1.9811 | 2.1821 | 1.7536 | 1.8631 | 1.7679 | 1.7009 |
| D | 1.2649 | 1.2504 | 1.6714 | 1.703 | 1.2489 | 1.3418 | 0.8885 | 0.9864 | 0.9552 | 0.9643 |
| E | 0.5966 | 0.6148 | 0.8319 | 0.9243 | 0.6348 | 0.6621 | 0.3714 | 0.4403 | 0.3928 | 0.4166 |
| F | 0.2681 | 0.2774 | 0.3728 | 0.419 | 0.2898 | 0.2986 | 0.1667 | 0.2001 | 0.1851 | 0.1931 |
| G | 0.1589 | 0.1475 | 0.1692 | 0.178 | 0.1513 | 0.166 | 0.1023 | 0.1171 | 0.1076 | 0.1142 |
| H | 0.0725 | 0.07 | 0.0714 | 0.0737 | 0.072 | 0.0747 | 0.0714 | 0.0768 | 0.0806 | 0.0734 |

| | 2-H3 | | 5-D7 | | Ab18 | |
|---|---|---|---|---|---|---|
| A | 3.5402 | 3.5486 | 3.2889 | 3.3281 | 2.288 | 2.3394 |
| B | 3.0076 | 3.0135 | 2.6347 | 2.66 | 1.1352 | 1.2038 |
| C | 2.4923 | 2.578 | 1.8413 | 1.84 | 0.5058 | 0.5265 |
| D | 1.7786 | 1.7678 | 1.0517 | 1.0929 | 0.2432 | 0.2399 |
| E | 0.9525 | 0.9749 | 0.4927 | 0.4684 | 0.1247 | 0.1345 |
| F | 0.44 | 0.4441 | 0.2196 | 0.2178 | 0.0837 | 0.0885 |
| G | 0.2294 | 0.2096 | 0.1256 | 0.1277 | 0.0746 | 0.075 |
| H | 0.0752 | 0.0748 | 0.0765 | 0.0771 | 0.0706 | 0.072 |

Cells of clone 5-G4, 2-C7, 5-E6, 5-G9, 2-H3, 5-D7 and 5-G6 were cultured in 30 ml of selective medium in a 125 ml shake flask under standard conditions (37° C., 5% CO₂, 130 rpm/minute) for 3 passages. For expression assay, when the cells were in good condition in the shake flask, a 30 ml fed-batch culture was carried out. Media supplement was added on D3, D5, D7 and D9 respectively and the glucose was monitored throughout until the cell viability reduced below 50%. The supernatants of the culture were collected and purified. The purification results are shown in FIG. 9.

Sample information is shown in Table 9.

TABLE 9

| Anti- | Concen- | | | | |
|---|---|---|---|---|---|
| body | tration | Volume | Quantity | Yield | Yield |
| 5-E6 | 4.49 mg/ml | 1.63 ml/vial | 7 vials | 51.23 mg/30 ml | 1.70 g/L |
| 5-G4 | 7.13 mg/ml | 1.64 ml/vial | 7 vials | 81.86 mg/30 ml | 2.72 g/L |
| 2-H3 | 3.91 mg/ml | 1.67 ml/vial | 7 vials | 45.71 mg/30 ml | 1.52 g/L |
| 5-D7 | 4.63 mg/ml | 1.55 ml/vial | 6 vials | 43.06 mg/30 ml | 1.44 g/L |
| 2-C7 | 3.64 mg/ml | 1.63 ml/vial | 7 vials | 41.53 mg/30 ml | 1.38 g/L |
| 5-G6 | 5.07 mg/ml | 1.65 ml/vial | 7 vials | 58.56 mg/30 ml | 1.95 g/L |
| 5-G9 | 6.14 mg/ml | 1.61 ml/vial | 7 vials | 69.20 mg/30 ml | 2.31 g/L |

### (5) Identification of Humanized Anti-Periostin Antibodies

Based on the expression and ELISA results, three stable transfected cell lines (5-G4, 5-G6 and 5-G9) were selected and used for WB detection to verify the binding specificity.

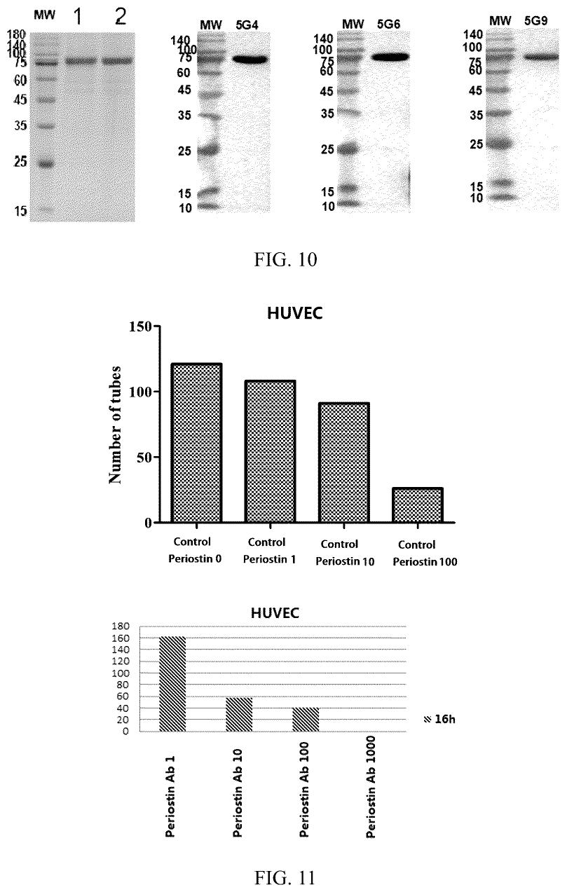

Experimental conditions: 1 μg of antigen, primary antibody (dilution at 1:1000), 1 hour of incubation, secondary antibody (dilution at 1:10000), 45 minutes of incubation. The results are shown in FIG. 10.

As shown in FIG. 10, three stable transfected lines (5-G4, 5-G6 and 5-G9) specifically bound to periostin protein.

### 4. Anti-Fibrotic Effect of the Humanized Anti-Periostin Antibody

### 4.1 Method

The antibody from stable transgenic cell line 5-G6 was used for transwell migration assay and for studying the number of migrated cells of fibroblasts, in vivo and in vitro. The fibroblast migration in mice was analyzed by immunohistochemical technology, and the histopathological changes in mice were detected by HE and Masson staining, the area ratio of fibrotic tissue was also studies. The mRNA expression of type I collagen (Col1a1 and Col1a2) and type III collagen (Col3a1) in the mouse model of fibrosis was detected by the real-time fluorescence quantitative polymerase chain reaction (PCR). The experiments included the conditioned medium containing periostin+control IgG as the IgG control group, and the conditioned medium containing periostin+humanized periostin antibody recorded as the Postn nAb group.

The area ratio of fibrotic tissue, the number of positive infiltrating fibroblasts, the ratio of fibroblasts and the median number of migrated fibroblasts were analyzed using Wilcoxon rank-sum test.

Finally, the experimental data was processed using SPSS Statistical Software, and variance between groups was analyzed by ANOVA, with significance defined as p<0.05 in all statistical analysis.

### 4.2 Results

4.2.1 In the transwell migration assay, the number of migrated fibroblasts in the blank control group was much larger than that in the Postn nAb group, which means that the Postn nAb is able to effectively inhibit the migration of fibroblasts and also confirmed the effectiveness of Postn nAb in vitro.

4.2.2 At 14 dpi, the immunohistological analysis results showed that Postn nAb reduced the number of infiltrating fibroblasts compared to the blank control.

4.2.3 The area ratio of fibrotic tissue at 28 dpi showed a significant reduction in fibrotic tissue in mice of Postn nAb group compared to the blank control.

4.2.4 The mRNA expression of collagen type I and collagen type III in mice of Postn nAb group was significantly reduced compared to the blank control group (P<0.05).

### CONCLUSION

A humanized anti-periostin monoclonal antibody is successfully produced by the present invention, and the humanized anti-periostin monoclonal antibody was able to specifically bind to periostin protein in vivo, thereby effectively inhibiting the fibrosis in vivo, so the humanized anti-periostin monoclonal antibody has the potential to be a novel medicament for the treatment of retinal fibrosis.

### 5. Activity of Humanized Periostin Monoclonal Antibody In Vitro

### 5.1 Tube Formation Inhibition Assay

HUVEC cells were plated at 50,000 cells/well (24 h) in a 12-well plate, cultured at 5% CO₂, 37° C. overnight in vitro, and then treated with the humanized periostin monoclonal antibody for 16 hours. The tube formation assay was performed. The cells were observed and photographed at desired time points. The statistical analysis results of the experimental data were shown in Table 10 and FIG. 11.

TABLE 10

| | Number | | Number |
|---|---|---|---|
| HUVEC | of tubes | HUVEC | of tubes |
| Group (μg/mL) | 16 h | Group (μg/mL) | 16 h |
| Control periostin 0 | 121 | periostin antibody 1 | 162 |
| Control periostin 1 | 108 | periostin antibody 10 | 57 |
| Control periostin 10 | 91 | periostin antibody 100 | 41 |
| Control periostin 100 | 26 | periostin antibody 1000 | 2 |

As can be seen from the above table, the number of tubes was reduced with increasing antibody concentration, and the tube formation inhibition rate by the humanized monoclonal antibody was greater than that by murine monoclonal antibody at the same concentration, which confirms that the above humanized periostin monoclonal antibody is bale to inhibit the tube formation of human vascular endothelial cells.

### 5.2 Cultivation of RPE Cells In Vitro and Grouping for Cell Migration Assay, Cell Invasion Assay and Cell Scratch Assay

a. Blank control group (Control): without recombinant periostin protein or antibody
b. Periostin group: with only periostin protein
c. Control IgG group: with periostin+control IgG
d. Periostin antibody group: with periostin+Postn nAb

### Postn nAb: The Humanized Anti-Periostin Antibody Produced by the Present Invention

### 5.2.1 Cell Migration Assay

### Transwell Assay

(1) Cells were centrifuged and then the culture medium was discarded. The cells were washed 1-2 times with PBS, resuspended in serum-free DMEM medium, and the cell density was adjusted to 1×10⁵ cells/ml.

(2) 200 μl of cell suspension was added to the transwell chamber, 600 μl/well of medium containing 10% FBS was added the lower chamber of a 24-well plate and incubated at 37° C. for 16-24 hours.

(3) The 24-well plate was removed, the medium in the transwell chamber was discarded, and the transwell chamber was washed twice with PBS.

(4) Cells were fixed with 4% PFA for 10 minutes and washed twice with PBS.

(5) The cells were subjected to Crystalline Violet staining for 10 minutes, washed twice with PBS.

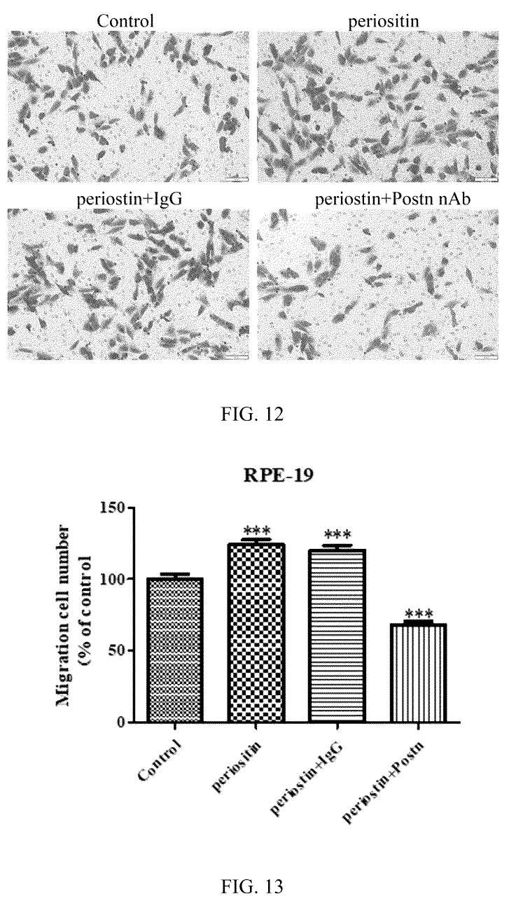

(6) Cell migration was observed under the microscope (FIG. 12), and 3 random microscope fields were imaged, counted and plotted with the Image J software (FIG. 13).

Experimental results are shown in Table 11.

TABLE 11

| | Migration cell number | | | average | % of control | | | AVERAGE |
|---|---|---|---|---|---|---|---|---|
| Control | 133 | 142 | 139 | 138.00 | 96.38% | 102.90% | 100.72% | 100.00% |
| Periositin | 177 | 168 | 168 | 171.00 | 128.26% | 121.74% | 121.74% | 123.91% |
| Periositin + IgG | 161 | 167 | 170 | 166.00 | 116.67% | 121.01% | 123.19% | 120.29% |
| Periositin + Postn nAb | 94 | 90 | 97 | 93.67 | 38.12% | 65.22% | 70.29% | 67.87% |

As shown in the experimental results, the ratio and number of migrated cells in the periostin-treated group were significantly increased (P<0.001), while those in the periostin+Postn nAb treated group were significantly reduced (P<0.001), both compared to the control group (Control). The periostin+IgG treated group had slightly less cell migration than the periostin-treated group. It demonstrates that the humanized periostin monoclonal antibody is able to inhibit migration of RPE cells induced by periostin.

### 5.2.2 Cell Invasion Assay

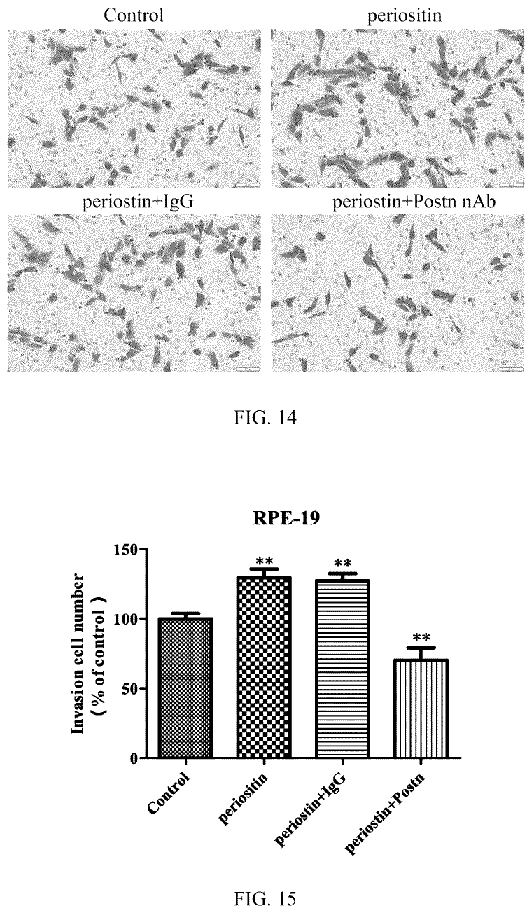

The medium containing FBS was added to the lower chamber, and a certain concentration of cell suspension was added to the transwell chamber pre-coated with Matrigel matrix, incubated at 37° C. and 5% CO₂ in an incubator for 48 hours, then observed and imaged (FIG. 14). The number of migrated cells was counted (FIG. 15) and results are shown in Table 12.

TABLE 12

| | Invasion cell number | | | Average | % of control | | | AVERAGE |
|---|---|---|---|---|---|---|---|---|
| Control | 78 | 84 | 80 | 80.67 | 96.69% | 104.13% | 99.17% | 100.00% |
| periositin | 100 | 103 | 110 | 104.33 | 123.97% | 127.69% | 136.36% | 129.34% |
| periostin + IgG | 106 | 104 | 98 | 102.67 | 131.40% | 128.93% | 121.49% | 127.27% |
| periostin + Postn nAb | 52 | 65 | 53 | 56.67 | 64.46% | 80.58% | 65.70% | 70.25% |

As shown in the experimental results, compared to the blank control group, the cell migration rate and the number of invasive cells in the periostin-treated group were significantly increased (P<0.01), while that in the periostin+Postn nAb-treated group were significantly reduced (P<0.01). The periostin+IgG treated group had slightly less cell migration than the periostin-treated group. It is showed that the humanized periostin monoclonal antibody has an inhibitive effect on periostin.

### 5.2.3 Cell Scratch Assay

(1) After the cells were grouped as described above, scratches were made using a 10 μl tip vertical to the plate, and the width of each scratch was same as possible.

The cell culture medium was removed, the plate was rinsed with PBS three times to wash away the cell debris resulting from the scratches, and the corresponding serum-free medium was added.

(3) The plate was placed in an incubator and photographed at 0, 6, 24 and 48 hours.

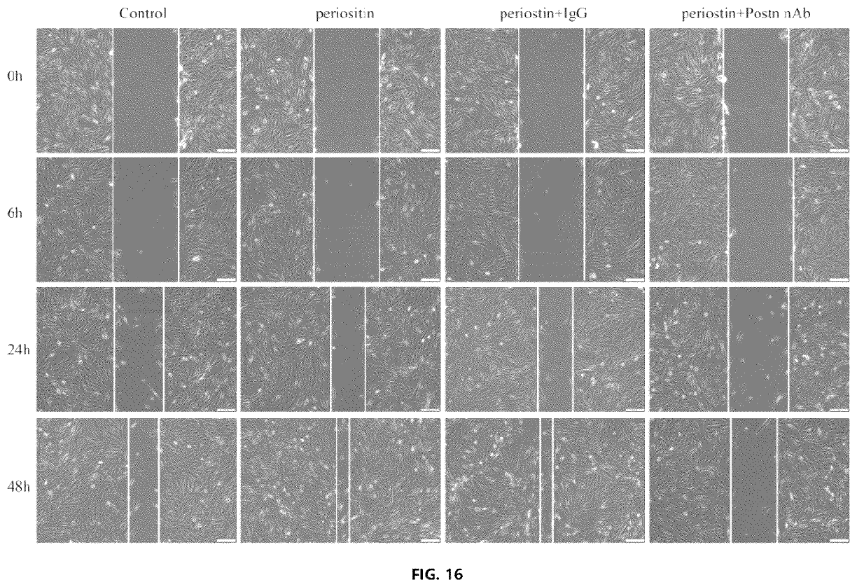

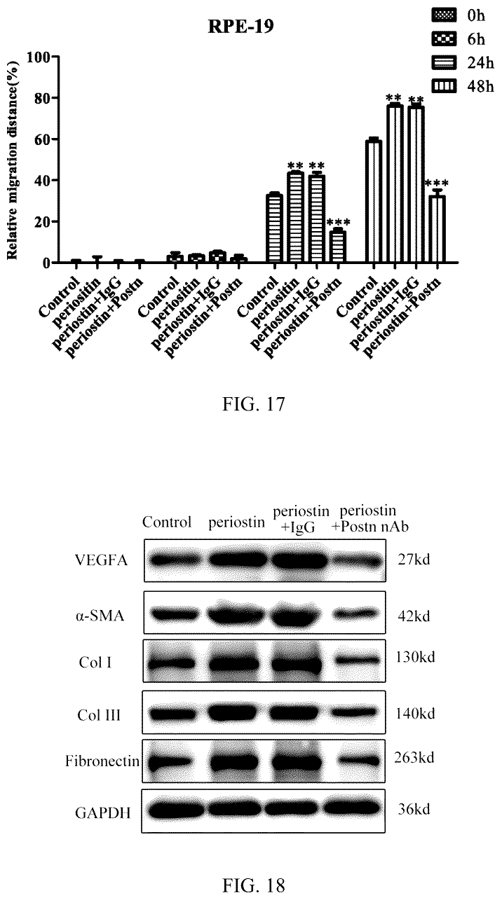

The data collected from pictures were analyzed to obtain experimental results (FIGS. 16-17).

Experimental results and analysis: After the cells were grouped as described above, scratches were made using a 10 μl tip vertical to the plate. The plate was placed in an incubator and photographed at 0, 6, 24 and 48 hours. Results are shown in Table 13 and Table 14.

TABLE 13

| | Distance (μm) | | | | | | | | | | | | |
|---|---|---|---|---|---|---|---|---|---|---|---|---|---|
| | 0 h | | | Average | 6 h | | | 24 h | | | 48 h | | |
| Control | 368.51 | 358.02 | 370.85 | 365.793 | 359.18 | 341.69 | 362.68 | 243.73 | 240.23 | 256.56 | 138.78 | 159.77 | 153.94 |
| periositin | 377.84 | 368.51 | 342.86 | 363.070 | 353.35 | 352.19 | 347.52 | 201.75 | 205.25 | 209.91 | 88.63 | 78.13 | 94.46 |
| periostin + | 359.18 | 362.68 | 351.02 | 357.627 | 338.19 | 345.19 | 338.19 | 206.41 | 219.24 | 197.08 | 77.3 | 88.63 | 97.79 |
| IgG | | | | | | | | | | | | | |
| periostin + | 358.02 | 369.68 | 360.35 | 362.683 | 359.18 | 344.02 | 364.34 | 313.7 | 297.38 | 316.03 | 267.06 | 246.06 | 226.24 |
| Postn nAb | | | | | | | | | | | | | |

TABLE 14

| | Relative migration distance (%) | | | | | | | | | | | |
|---|---|---|---|---|---|---|---|---|---|---|---|---|
| | 0 h | | | 6 h | | | 24 h | | | 48 h | | |
| Control | −0.74% | 2.13% | −1.38% | 1.81% | 6.59% | 0.85% | 33.37% | 34.33% | 29.86% | 62.06% | 56.32% | 57.92% |
| periositin | −4.07% | −1.50% | 5.57% | 2.68% | 3.00% | 4.28% | 44.43% | 43.47% | 42.18% | 75.59% | 78.48% | 73.98% |
| periostin + IgG | −0.43% | −1.41% | 1.85% | 5.43% | 3.48% | 5.43% | 42.28% | 38.70% | 44.89% | 78.39% | 75.22% | 72.66% |
| periostin + Postn nAb | 1.29% | −1.93% | 0.64% | 0.97% | 5.15% | −0.46% | 13.51% | 18.01% | 12.86% | 26.37% | 32.16% | 37.62% |

As shown in the experimental results, at 48 hours, the relative migration distance of the cells in the periostin-treated group was significantly increased (P<0.01), while that in the periostin+Postn nAb-treated group were significantly reduced (P<0.01), both compared to the blank control group. The periostin+IgG treated group had slightly less cell migration than the periostin-treated group. It is showed that the humanized periostin monoclonal antibody has an inhibitive effect on migration of RPE cells induced by periostin.

### 5.3 Results of Western Blot

The expressions of VEGFA, α-SMA, Col I, Col III and Fibronectin proteins of RPE cells in different groups were detected. Results are shown in Tables 15-19.

VEGFA (vascular endothelial growth factor A) induces angiogenesis and promotes vascular regeneration.

α-SMA (α-Smooth muscle actin) is present in the vascular wall, intestinal mucosal muscularis, muscularis propria, and various tissue mesenchyme, highly expressed in myofibroblasts and myoepithelial cells, and has been shown to increase with increasing fibrosis in studies of organ fibrosis.

Col I (collagen type I) and Col III (collagen type III) promote the proliferation of fibroblasts in the dermis and are closely associated with the wound repair process and the quality of skin injury. They are mainly found in connective tissues such as skin, tendons, ligaments and blood vessels, and are components of the extracellular matrix, which plays a role in supporting the organs and protecting the body, and is also involved in cell attachment and migration.

Fibronectin promotes the growth of adherent cells, affects cell adhesion, cell migration, tumor metastasis, and embryonic development, growth and differentiation, etc.

TABLE 15

| Group | VEGFA | | | GAPDH | | | VEGFA:GAPDH | | | Average | Relative value | | |
|---|---|---|---|---|---|---|---|---|---|---|---|---|---|
| Control | 581015 | 556895 | 602545 | 1053645 | 1075620 | 1035685 | 0.55 | 0.52 | 0.58 | 0.55 | 1.00 | 0.94 | 1.06 |
| periostin | 1082105 | 1036598 | 1125416 | 1030138 | 1012536 | 1053265 | 1.05 | 1.02 | 1.07 | 1.05 | 1.91 | 1.86 | 1.94 |
| periostin + | 1059098 | 992547 | 1076853 | 956898 | 932658 | 986852 | 1.11 | 1.06 | 1.09 | 1.09 | 2.01 | 1.93 | 1.98 |
| IgG | | | | | | | | | | | | | |
| periostin + | 381691 | 426452 | 402541 | 1019821 | 998652 | 1036895 | 0.37 | 0.43 | 0.39 | 0.40 | 0.68 | 0.78 | 0.71 |
| Postn nAb | | | | | | | | | | | | | |

TABLE 16

| Group | α-SMA | | | GAPDH | | | α-SMA:GAPDH | | | Average | Relative value | | |
|---|---|---|---|---|---|---|---|---|---|---|---|---|---|
| Control | 519129 | 536588 | 557410 | 1053645 | 1075620 | 1035685 | 0.49 | 0.50 | 0.54 | 0.51 | 0.97 | 0.98 | 1.06 |
| periostin | 917194 | 938752 | 897452 | 1030138 | 1012536 | 1053265 | 0.89 | 0.93 | 0.85 | 0.89 | 1.75 | 1.82 | 1.67 |
| periostin + | 873202 | 845685 | 901254 | 956898 | 932658 | 986852 | 0.91 | 0.91 | 0.91 | 0.91 | 1.79 | 1.78 | 1.79 |
| IgG | | | | | | | | | | | | | |
| periostin + | 320513 | 302125 | 356420 | 1019821 | 998652 | 1036895 | 0.31 | 0.30 | 0.34 | 0.32 | 0.62 | 0.59 | 0.67 |
| Postn nAb | | | | | | | | | | | | | |

TABLE 17

| Group | ColI | | | GAPDH | | | Col:GAPDH | | | Average | Relative value | | |
|---|---|---|---|---|---|---|---|---|---|---|---|---|---|
| Control | 501679 | 535627 | 545896 | 1053645 | 1075620 | 1035685 | 0.48 | 0.50 | 0.53 | 0.50 | 0.95 | 1.00 | 1.05 |
| periostin | 847771 | 865986 | 893568 | 1030138 | 1012536 | 1053265 | 0.82 | 0.86 | 0.85 | 0.84 | 1.64 | 1.71 | 1.70 |
| periostin + | 895815 | 850241 | 876231 | 956898 | 932658 | 986852 | 0.94 | 0.91 | 0.89 | 0.91 | 1.87 | 1.82 | 1.77 |
| IgG | | | | | | | | | | | | | |
| periostin + | 298207 | 275410 | 315221 | 1019821 | 998652 | 1036895 | 0.29 | 0.28 | 0.30 | 0.29 | 0.58 | 0.55 | 0.61 |
| Postn nAb | | | | | | | | | | | | | |

TABLE 18

| Group | Col III | | | GAPDH | | | Col III:GAPDH | | | Average | Relative value | | |
|---|---|---|---|---|---|---|---|---|---|---|---|---|---|
| Control | 653408 | 685412 | 613154 | 1053645 | 1075620 | 1035685 | 0.62 | 0.64 | 0.59 | 0.62 | 1.01 | 1.03 | 0.96 |
| periostin | 1041617 | 1085265 | 1102458 | 1030138 | 1012536 | 1053265 | 1.01 | 1.07 | 1.05 | 1.04 | 1.64 | 1.74 | 1.70 |
| periostin + | 954461 | 975521 | 1003545 | 956898 | 932658 | 986852 | 1.00 | 1.05 | 1.02 | 1.02 | 1.62 | 1.70 | 1.65 |
| gG | | | | | | | | | | | | | |
| periostin + | 399965 | 421352 | 455482 | 1019821 | 998652 | 1036895 | 0.39 | 0.42 | 0.44 | 0.42 | 0.64 | 0.68 | 0.71 |
| Postn nAb | | | | | | | | | | | | | |

TABLE 19

| Group | Fibronectin | | | GAPDH | | | Fibronectin:GAPDH | | | Average | Relative value | | |
|---|---|---|---|---|---|---|---|---|---|---|---|---|---|
| Control | 597721 | 622547 | 566214 | 1053645 | 1075620 | 1035685 | 0.57 | 0.58 | 0.55 | 0.56 | 1.01 | 1.03 | 0.97 |
| periostin | 915085 | 936542 | 956223 | 1030138 | 1012536 | 1053265 | 0.89 | 0.92 | 0.91 | 0.91 | 1.57 | 1.64 | 1.61 |
| periostin + | 944392 | 968725 | 923574 | 956898 | 932658 | 986852 | 0.99 | 1.04 | 0.94 | 0.99 | 1.75 | 1.84 | 1.66 |
| IgG | | | | | | | | | | | | | |
| periostin + | 337385 | 356854 | 387931 | 1019821 | 998652 | 1036895 | 0.33 | 0.36 | 0.37 | 0.35 | 0.59 | 0.63 | 0.66 |
| Postn nAb | | | | | | | | | | | | | |

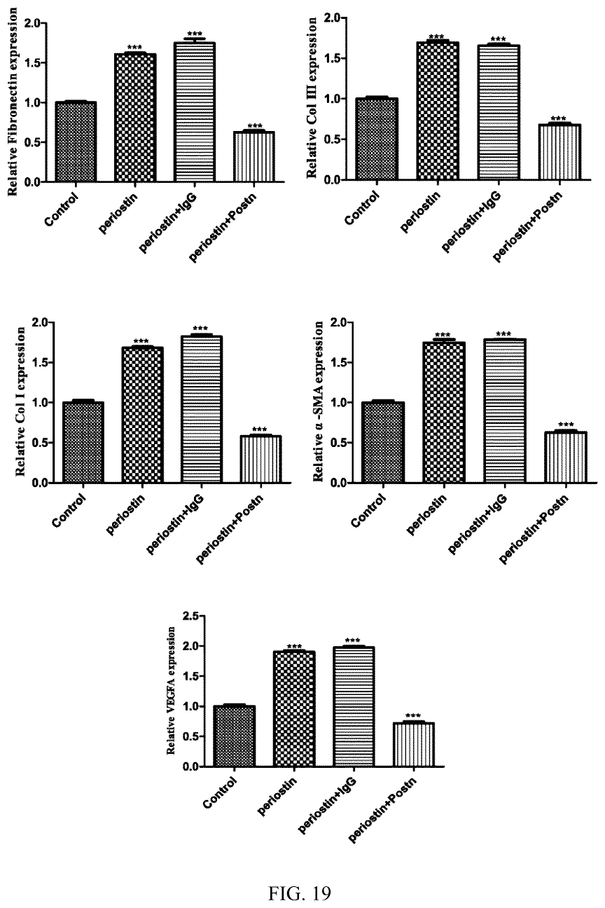

As shown in the experimental results (FIGS. 18-19), there are statistically significant difference in the protein expressions between periostin-treated, periostin+IgG treated, and periostin+Postn nAb treated groups and the blank control group (Control) (P<0.001). Compared to the blank control group (Control), the expression of all tested proteins in periostin-treated groups were significantly increased (P<0.001), while the expression of proteins (VEGFA, α-SMA, Col I, Col III and Fibronectin) in periostin+Postn nAb-treated group were all significantly reduced (P<0.001), indicating that periostin monoclonal antibody can inhibit the expression of proteins VEGFA, α-SMA, Col I, Col III, and Fibronectin.

### 6. Real-Time PCR, WB, and IF Assays for HUVEC Cells In Vitro

Periostin promotes retinal neovascularization and fibrosis through the integrin-focal adhesion kinase (FAK)-phosphatidylinositol 3-kinase (PI3K)-protein kinase B (AKT/PKB) signaling. In order to confirm the therapeutic effect of the humanized periostin monoclonal antibody on vitreoretinal fibrosis, real-time PCR, immunofluorescence and Western blot were used to measure the expression of mRNA and protein of integrin (αvβ3 and αvβ5), vascular endothelial growth factor receptor-2 (VEGFR-2), FAK, PI3K and AKT on the membrane of the human vascular endothelial cells.

The HUVEC cells were cultured in vitro and grouped.
a. Blank control group (Control) (without recombinant periostin protein or antibody)
b. Periostin group (with only periostin protein)
c. Control IgG group (with periostin+control IgG)
d. periostin antibody group (with periostin+Postn nAb)

### Postn nAb: The Humanized Periostin Antibody Produced by the Present Invention

Human periostin protein was incubated with human periostin antibody for 1 hour and then added to the cells for 24 hours for the following assays, in which the concentration of control IgG used was the same as that of the humanized anti-periostin monoclonal antibody.

### 6.1 Real-Time PCR Assay

The mRNA expressions of integrins (αvβ3 and αvβ5), VEGFR-2, FAK, PI3K, p-PI3K, AKT and p-AKT on the membrane of the vascular endothelial cells were measured.

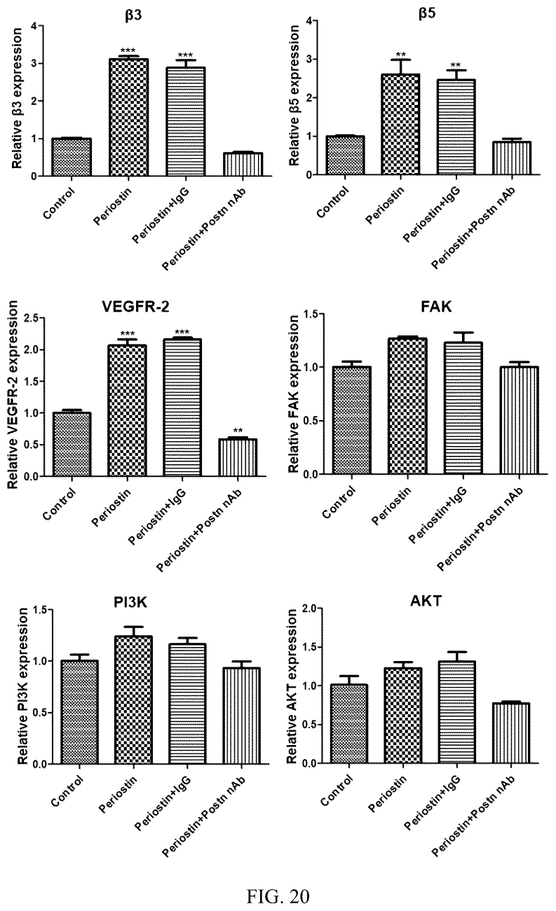

As shown in FIG. 20, compared to the blank control group, the gene expression of αvβ3, αvβ5, VEGFR-2, FAK, PI3K, p-PI3K, AKT and p-AKT in the periostin-treated group were all increased, while those in the periostin+Postn nAb-treated group were all decreased, in particular the mRNA expression of VEGFR-2 was significantly lower than that of the blank control group (P<0.001), indicating that the humanized periostin monoclonal antibody can inhibit the mRNA expression of VEGFR-2, FAK, PI3K, p-PI3K, AKT and p-AKT.

6.2 Detection of Protein Expressions of αvβ3, αvβ5, VEGFR-2, FAK, PI3K, p-PI3K, AKT and p-AKT in HUVEC Cells by Western Blot

Normal cultured HUVEC cells were plated at 10⁶ cells/dish (6 cm dish) and cultured at 5% CO₂, 37° C., overnight. After adhesion, the cells were treated according to the grouping. Total protein was subjected to content detection using the BCA kit, SDS-PAGE, followed by membrane transferring, blocking with blocking buffer, washing, incubating with the primary antibody overnight in a refrigerator at 4° C., washing, incubating with the secondary antibody at room temperature on a shaker for 1 hour, and performing color development. The gray-scale value ratio of the tested proteins to the reference protein under the same conditions in each group was used as the relative expression of the tested proteins.

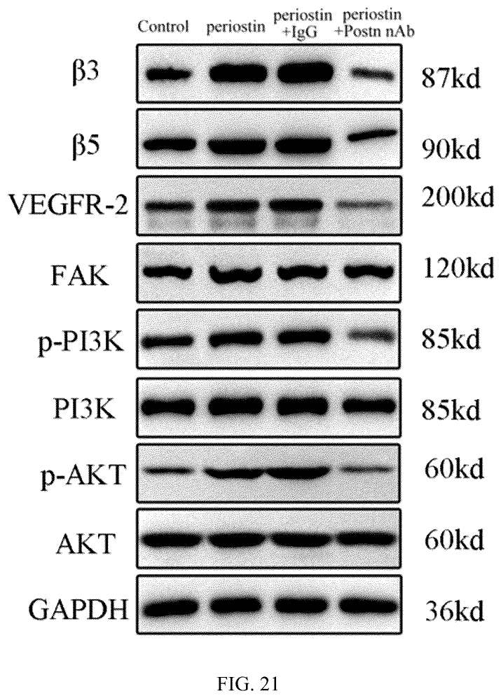

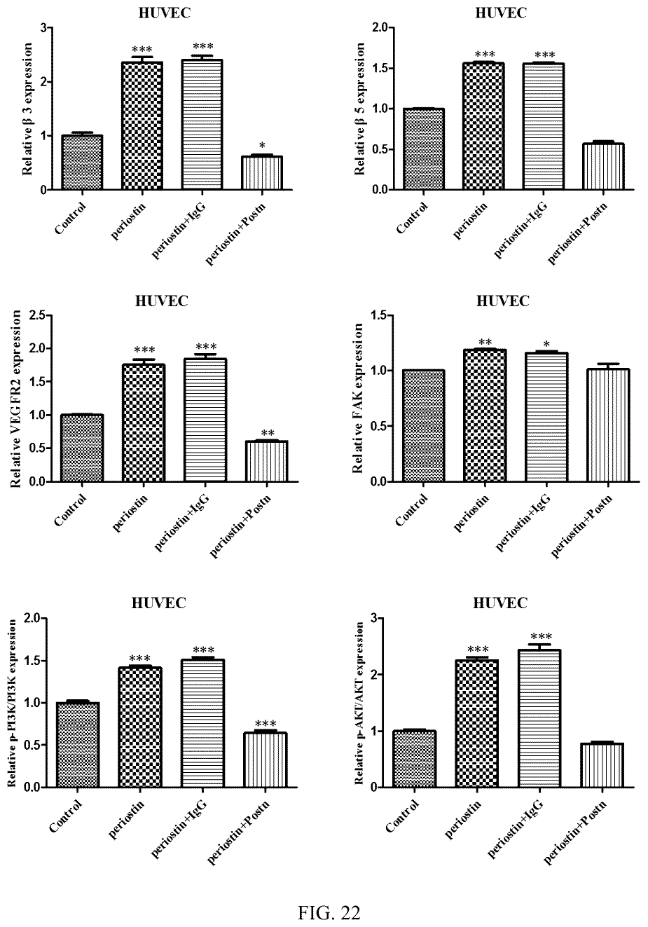

The expression and grey scale analysis of proteins in HUVEC cells of different groups are shown in FIGS. 21-22. As shown in the experimental results, compared to the blank control group, the expressions of αvβ3, αvβ5, VEGFR-2, FAK, PI3K, p-PI3K, AKT and p-AKT in the periostin-treated group were increased, while those in the periostin+Postn nAb treated group were reduced. The expression of these protein in the periostin+Postn nAb treated group were all lower than those in periostin treated group, indicating that the humanized periostin monoclonal antibody has an inhibitive effect on periostin.

6.3 Detection of the Expression of Protein αvβ3, αvβ5, VEGFR-2 and FAK in HUVEC Cells by IF

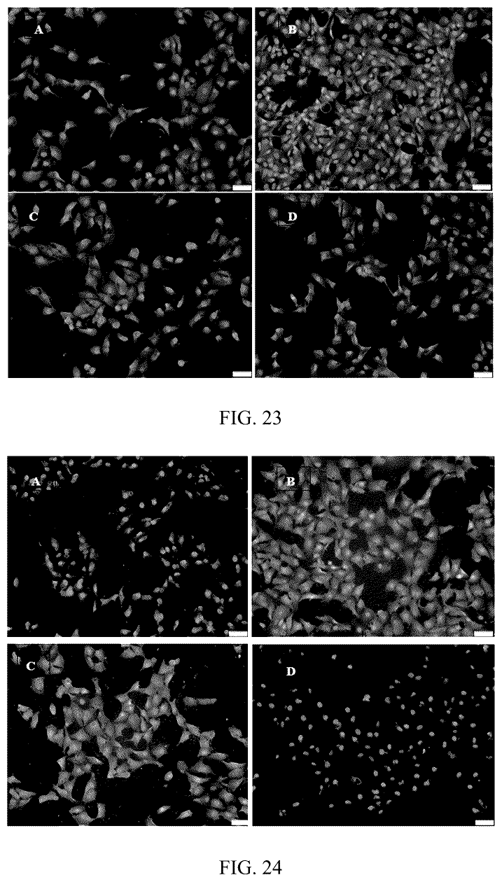

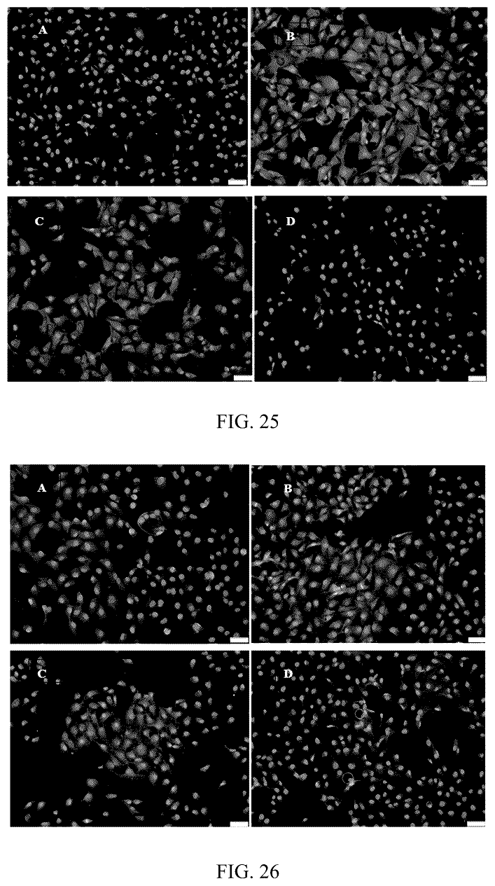

The coverslips where cells had grew were washed with PBS in the culture plate, fixed with 4% paraformaldehyde, permeabilized with 0.5% Triton X-100 for 20 minutes at room temperature. The coverslips were incubated with primary antibody in wet box at 4° C. overnight and then incubated with fluorescent secondary antibody in wet box at 20-37° C. for 1 hour. The cell nuclei were counterstained by dropwise addition of DAPI, and the coverslips were mounted and observed under fluorescence microscope (FIGS. 23-26).

Under the fluorescence microscopy, it was seen that periostin protein was expressed in the cytoplasm of HUVEC cells. αvβ3, αvβ5, VEGFR-2 and FAK showed weak fluorescence in the cell cytoplasm in the blank control group and strong fluorescence in the periostin-treated group. The fluorescence signals of the cells in the periostin+Postn nAb-treated group were significantly weaker than those in the periostin-treated group, and were no statistically significant compared to those in the blank control group, indicating that the humanized periostin monoclonal antibody can inhibit the action of periostin by blocking the integrin-FAK-PI3K-AKT/PKB signaling, thereby inhibiting retinal fibrosis.

The above embodiments are only preferred embodiments of the present invention. It should be noted that, for those skilled in the art, other improvements and modifications may be further made without departing from the principle of the present invention, and these improvements and modifications should also be deemed as falling into the protection scope of the present invention.

1. A humanized anti-periostin monoclonal antibody comprising a heavy chain variable region and a light chain variable region, wherein the heavy chain variable region comprises an amino acid sequence set forth in SEQ ID NO: 1, and the light chain variable region comprises an amino acid sequence set forth in SEQ ID NO: 2.

2. The humanized anti-periostin monoclonal antibody according to claim 1, which comprises a heavy chain constant region and a light chain constant region, wherein the heavy chain constant region is a heavy chain constant region of human IgG1, and the light chain constant region is a light chain constant region of kappa chain.

3. The humanized anti-periostin monoclonal antibody according to claim 1, which comprises a heavy chain and a light chain, wherein the amino acid sequence of the heavy chain is set forth in SEQ ID NO: 3, and the amino acid sequence of the light chain is set forth in SEQ ID NO: 4.

4. A nucleic acid encoding the humanized anti-periostin monoclonal antibody according to claim 1.

5. The nucleic acid according to claim 4, wherein the sequence of the nucleic acid encoding the heavy chain of the humanized anti-periostin monoclonal antibody is SEQ ID NO: 5, and the sequence of the nucleic acid encoding the light chain of the humanized anti-periostin monoclonal antibody is SEQ ID NO: 6.

6. The nucleic acid according to claim 5, wherein the sequence of the nucleic acid encoding the heavy chain of the humanized anti-periostin monoclonal antibody is SEQ ID NO: 7, and the sequence of the nucleic acid encoding the light chain of the humanized anti-periostin monoclonal antibody is SEQ ID NO: 8.

7. An expression vector comprising the nucleic acid according to claim 4.

8. The expression vector according to claim 7, wherein a backbone vector of the expression vector is PATX-GS2.

9. A host cell transformed or transfected with the expression vector according to claim 8.

10. A method for producing the humanized anti-periostin monoclonal antibody according to claim 1, comprising culturing a host cell transformed or transfected with an expression vector comprising a nucleic acid encoding the humanized anti-periostin monoclonal antibody and inducing the expression of the humanized anti-periostin monoclonal antibody.

11. A method of treating tissue fibrosis and/or a malignant tumor comprising administering the humanized anti-periostin monoclonal antibody according to claim 1 to a subject in need thereof.

12. The method according to claim 11, wherein the tissue fibrosis is retinal fibrosis.

13. A medicament for the treatment of tissue fibrosis and/or a malignant tumor, comprising the humanized anti-periostin monoclonal antibody according to claim 1.

14. A kit for diagnosing tissue fibrosis and/or a malignant tumor, comprising the humanized anti-periostin monoclonal antibody according to claim 1.


SEQUENCE LISTING (38 sequences)

SEQ ID NO: 1 - protein, 117 aa, synthetic construct
EVQLVQSGAEVKKPGESLKISCKASGYSFTDYFMNWVRQMPGKGLEWIGRINPYSGDTLYNQRLQGQVTLSADKSISTAYLQLSSLKASDTAMYYCGRSGVSGLDYWGQGTLVTVSS

SEQ ID NO: 2 - protein, 106 aa, synthetic construct
DIQLTQSPSSLSASVGDRVTITCSASSSASYMHWYQQKPGKAPKNWIYDTSKLASGVPSRFSGSGSGTDYTLTISSLQPEDAATYYCQQWSSNPPTFGGGTKVEIK

SEQ ID NO: 3 - protein, 466 aa, synthetic construct
MKHLWFFLLLVAAPRWVLSEVQLVQSGAEVKKPGESLKISCKASGYSFTDYFMNWVRQMPGKGLEWIGRINPYSGDTLYNQRLQGQVTLSADKSISTAYLQLSSLKASDTAMYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 233 aa, synthetic construct
MVLQTQVFISLLLWISGAYGDIQLTQSPSSLSASVGDRVTITCSASSSASYMHWYQQKPGKAPKNWIYDTSKLASGVPSRFSGSGSGTDYTLTISSLQPEDAATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1401 bp, synthetic construct
  source 1..1401: mol_type = other DNA
atgaagcacctgtggttcttcctgctgctggtggctgctcctaggtgggtgctgagcgaggtgcagctggtgcagagcggcgccgaggtgaagaagcccggcgagagcctgaagatctcctgtaaggcttccggctactccttcaccgactactttatgaattgggtgcggcagatgcccggcaagggcctggagtggatcggcagaatcaatccttacagcggcgacaccctgtacaaccagcggctgcagggccaggtgaccctgtccgctgataagagcatctccaccgcctacctgcagctgtcctccctgaaggcctccgacaccgccatgtactactgtggcaggagcggcgtgagcggcctggactactggggccagggcaccctggtgaccgtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatga

SEQ ID NO: 6 - DNA, 702 bp, synthetic construct
  source 1..702: mol_type = other DNA
atggtgctgcagacccaggtgtttatcagcctgctgctgtggatcagcggcgcttacggcgacatccagctgacccagtccccctccagcctgagcgctagcgtgggcgaccgggtgaccatcacctgctccgcctccagctccgccagctacatgcactggtaccagcagaagcccggcaaggcccccaagaactggatctatgataccagcaagctggccagcggcgtgcccagcagattcagcggcagcggctccggcaccgattacaccctgaccatcagctccctgcagcccgaggatgctgccacctactactgccagcagtggtcctccaacccccctacctttggcggcggcaccaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctga

SEQ ID NO: 7 - DNA, 1424 bp, synthetic construct
  source 1..1424: mol_type = other DNA
gaattcgccgccaccatgaagcacctgtggttcttcctgctgctggtggctgctcctaggtgggtgctgagcgaggtgcagctggtgcagagcggcgccgaggtgaagaagcccggcgagagcctgaagatctcctgtaaggcttccggctactccttcaccgactactttatgaattgggtgcggcagatgcccggcaagggcctggagtggatcggcagaatcaatccttacagcggcgacaccctgtacaaccagcggctgcagggccaggtgaccctgtccgctgataagagcatctccaccgcctacctgcagctgtcctccctgaaggcctccgacaccgccatgtactactgtggcaggagcggcgtgagcggcctggactactggggccagggcaccctggtgaccgtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 8 - DNA, 725 bp, synthetic construct
  source 1..725: mol_type = other DNA
gaattcgccgccaccatggtgctgcagacccaggtgtttatcagcctgctgctgtggatcagcggcgcttacggcgacatccagctgacccagtccccctccagcctgagcgctagcgtgggcgaccgggtgaccatcacctgctccgcctccagctccgccagctacatgcactggtaccagcagaagcccggcaaggcccccaagaactggatctatgataccagcaagctggccagcggcgtgcccagcagattcagcggcagcggctccggcaccgattacaccctgaccatcagctccctgcagcccgaggatgctgccacctactactgccagcagtggtcctccaacccccctacctttggcggcggcaccaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 19 aa, synthetic construct
MKHLWFFLLLVAAPRWVLS

SEQ ID NO: 12 - protein, 20 aa, synthetic construct
MVLQTQVFISLLLWISGAYG

SEQ ID NO: 13 - protein, 106 aa, synthetic construct
DIVLTQSPAIMSASPGDKVTMTCSASSSASYMHWYQQKSGTSPKRWIYDTSKLASGVPARFSGSGSGTSYSLTISSMEAEDAATYYCQQWSSNPPTFGGGTKLEIK

SEQ ID NO: 14 - protein, 106 aa, synthetic construct
QIVLTQSPVIMSASPGDKVTMTCSASSSASYMHWYQQKSGTSPKRWIYDTSKLASGVPARFSGSGSGTSYSLTISSMEAEDAATYYCQQWSSNPPTFGGGTKLEIK

SEQ ID NO: 15 - protein, 117 aa, synthetic construct
EVQLQQSGPELVKPGASVRISCKASGYSFTDYFMNWVKQSHGRSLEWIGRINPYSGDTLYNQRLKGKATLTVDKSSSTAHMELLSLTSEDSAVYYCGRSGVSGLDYWGQGTSVTVSS

SEQ ID NO: 16 - protein, 5 aa, synthetic construct
SSASY

SEQ ID NO: 17 - protein, 9 aa, synthetic construct
QQWSSNPPT

SEQ ID NO: 18 - protein, 8 aa, synthetic construct
GYSFTDYF

SEQ ID NO: 19 - protein, 8 aa, synthetic construct
INPYSGDT

SEQ ID NO: 20 - protein, 10 aa, synthetic construct
GRSGVSGLDY

SEQ ID NO: 21 - DNA, 1424 bp, synthetic construct
  source 1..1424: mol_type = other DNA
gaattcgccgccaccatgaagcacctgtggtttttcctgctgctggtggccgcccccaggtgggttctgtcccaggtgcagctggtgcagagcggcgccgaggtgaagaagcctggcgcttccgtgaaggtgtcctgcaaggcttctggctatagctttaccgattatttcatgaactgggtgcggcaggctcctggccagggcttggagtggatcggcaggatcaacccttactccggcgacaccctgtataaccagaggctgaagggccgggccaccctgaccgtggataagagcatcagcaccgcttatatggagctgtcccggctgcggtccgacgacaccgctgtgtattactgcggcaggtccggcgtgtccggcctggattactggggccagggcaccctggtgaccgtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 22 - DNA, 1424 bp, synthetic construct
  source 1..1424: mol_type = other DNA
gaattcgccgccaccatgaagcacctgtggtttttcctgctgctggtggccgctcctcggtgggtgctgtcccaggtgcagctggtgcagagcggcgccgaggtgaagaagcctggcgcttccgtgaaggtgtcctgtaaggccagcggctacagcttcaccgactactttatgaactgggtgaggcaggctcctggccagggcctggagtggatcggcaggatcaacccctatagcggcgacaccctgtacaatcagaagctgcagggccgggtgaccatgaccgtggacaagtccatcagcaccgcttacatggagctgtcccggctgcggagcgacgataccgctgtgtattactgcggccggtccggcgtgagcggcttggattattggggccagggcaccctggtgaccgtgagctccgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 23 - DNA, 1424 bp, synthetic construct
  source 1..1424: mol_type = other DNA
gaattcgccgccaccatgaagcacctgtggtttttcctgctgctggtggccgccccccggtgggtgctgtcccaggtgcagctgcaggagagcggccctggcctggtgaagcccagcgagaccctgagcctgacctgcaccgcttccggctacagcttcaccgattacttcatgaactgggtgcggcagccccctggcaagggcttggagtggatcggcaggatcaacccttacagcggcgacaccctgtataatcagcggctgaagggcagggtgaccctgagcgtggataagagcaagaaccaggccagcctgaagctgagcagcgtgaccgctgccgataccgccgtgtattattgtggccggtccggcgtgagcggcctggattactggggccagggcaccctggtgaccgtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 24 - DNA, 1424 bp, synthetic construct
  source 1..1424: mol_type = other DNA
gaattcgccgccaccatgaagcacctgtggtttttcctgctgctggtggctgctcctcggtgggtgctgagccaggtgcagctgcaggagagcggccccggactggtgaagcctagcgagaccctgtccctgacctgcaccgccagcggctattcctttaccgattatttcatgaactggatccggcagccccctggcaagggcctggagtggatcggccggatcaacccctattccggcgataccctgtacaaccagcggctgaagtccagggtgaccctgagcgtggacaagtccaagaaccaggcttccctgaagctgtccagcgtgaccgctgctgataccgctgtgtactactgcggccggagcggcgtgtccggcttggattattggggccagggcaccctggtgaccgtgagcagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 25 - DNA, 1424 bp, synthetic construct
  source 1..1424: mol_type = other DNA
gaattcgccgccaccatgaagcacctgtggttctttctgctgctggtggccgcccccaggtgggttctgagccaggtgcagctgcaggagagcggccccggactggtgaagcctagcgagaccctgtccctgacctgcaccgtgtccggcggctccatcaccgattatttcatgaactggatcaggcagccccctggcaagggcctggagtggatcggccggatcaatccctatagcggcgacaccctgtacaatcagcggctgaagagcagggtgaccctgagcgtggataagtccaagaatcaggccagcctgaagctgtcctccgtgaccgccgccgacaccgctgtgtactactgcggccggtccggcgtgagcggcttggattactggggccagggcaccctggtgaccgtgtccagcgctagcaccaagggaccttctgtgttccctctggctccttcttctaagtccacttccggtggtacagcagctctgggttgtctggtgaaggattacttcccagaaccagtgactgtgtcctggaactccggagctctgacttctggagtgcatactttcccagcagtgctgcaatctagcggactgtactctctgtcttccgtggtgactgtgccttcttcttccctggggactcaaacttacatctgcaacgtgaaccacaagccctccaacaccaaggtggacaagaaggtggagccaaagagctgcgataagacccacacctgtccaccttgtccagctccagaactgctgggtgggccttctgtgtttctgttcccacctaagccaaaggataccctgatgatctctaggaccccagaagtgacctgtgtggtcgtcgatgtgtctcatgaagaccctgaagtgaagttcaactggtacgtggacggggtggaagtgcataacgcaaagaccaagcccagggaagagcaatacaactccacctacagggtggtctccgtcctgacagtcctgcatcaggattggctgaacggcaaggagtacaagtgcaaggtctccaataaagccctgcctgcccctatcgagaaaaccattagcaaagccaaaggccagcccagggagccccaggtctatacactgccccccagcagggaggagatgacaaaaaatcaggtcagcctgacatgcctggtcaaaggcttttatcccagcgacattgccgtcgagtgggagtccaatggccagcccgagaataattataaaacaacaccccccgtcctggacagcgacggcagcttttttctgtatagcaaactgacagtcgataaaagcaggtggcagcagggcaatgtcttttcctgcagcgtcatgcacgaggccctgcacaatcactatactcagaaaagcctgagcctgtcccccgggaaatgagcggccgc

SEQ ID NO: 26 - DNA, 1424 bp, synthetic construct; identical to SEQ ID NO: 7
  source 1..1424: mol_type = other DNA

SEQ ID NO: 27 - DNA, 725 bp, synthetic construct
  source 1..725: mol_type = other DNA
gaattcgccgccaccatggtgctgcagacccaggtgtttatctccctgctgctgtggatcagcggcgcctatggcgagatcgtgctgacccagtcccctgatttccagagcgtgacccctaaggagaaggtgaccatcacctgcagcgccagcagctccgccagctatatgcactggtaccagcagaagcccgaccagtcccctaagcggtggatctatgacaccagcaagctggctagcggcgtgcctagcaggttctccggcagcggcagcggcaccgactacaccctgaccatcaactccctggaggctgaggacgccgccacctattactgccagcagtggagctccaacccccctacctttggcggcggcaccaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 28 - DNA, 725 bp, synthetic construct
  source 1..725: mol_type = other DNA
gaattcgccgccaccatggtgctgcagacccaggtgtttatcagcctgctgctgtggatcagcggcgcctacggcgagatcgtgctgacccagtcccccgccaccctgtccctgtccccaggagagagggctaccctgagctgctccgccagctccagcgcctcctacatccactggtaccagcagaagcctggccaggcccctcggagatggatgtacgatacctccaagctggcctccggcatccccgccagattcagcggcagcggcagcggaaccgattacaccctgaccatcagctccctggagcctgaggacgccgccgtgtactactgccagcagtggagcagcaaccctcctaccttcggcggcggcaccaaggtggagatcaagcgtacggtggctgcaccttctgtgttcatcttccctccatctgatgagcagctgaagtctggaaccgcatctgtcgtctgtctgctgaacaacttttaccccagggaggctaaggtccaatggaaggtggacaacgccctgcagtctggtaatagccaggaaagcgtgaccgaacaggattccaaggactccacctactccctgtcctccacactgacactgagcaaagccgactatgaaaagcacaaagtgtatgcctgcgaggtcactcatcagggcctgtccagccccgtgactaaaagctttaataggggggagtgctgagcggccgc

SEQ ID NO: 29 - DNA, 725 bp, synthetic construct; identical to SEQ ID NO: 8
  source 1..725: mol_type = other DNA

SEQ ID NO: 30 - protein, 466 aa, synthetic construct
MKHLWFFLLLVAAPRWVLSQVQLVQSGAEVKKPGASVKVSCKASGYSFTDYFMNWVRQAPGQGLEWIGRINPYSGDTLYNQRLKGRATLTVDKSISTAYMELSRLRSDDTAVYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 466 aa, synthetic construct
MKHLWFFLLLVAAPRWVLSQVQLVQSGAEVKKPGASVKVSCKASGYSFTDYFMNWVRQAPGQGLEWIGRINPYSGDTLYNQKLQGRVTMTVDKSISTAYMELSRLRSDDTAVYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 466 aa, synthetic construct
MKHLWFFLLLVAAPRWVLSQVQLQESGPGLVKPSETLSLTCTASGYSFTDYFMNWVRQPPGKGLEWIGRINPYSGDTLYNQRLKGRVTLSVDKSKNQASLKLSSVTAADTAVYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 466 aa, synthetic construct
MKHLWFFLLLVAAPRWVLSQVQLQESGPGLVKPSETLSLTCTASGYSFTDYFMNWIRQPPGKGLEWIGRINPYSGDTLYNQRLKSRVTLSVDKSKNQASLKLSSVTAADTAVYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 466 aa, synthetic construct
MKHLWFFLLLVAAPRWVLSQVQLQESGPGLVKPSETLSLTCTVSGGSITDYFMNWIRQPPGKGLEWIGRINPYSGDTLYNQRLKSRVTLSVDKSKNQASLKLSSVTAADTAVYYCGRSGVSGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 466 aa, synthetic construct; identical to SEQ ID NO: 3

SEQ ID NO: 36 - protein, 233 aa, synthetic construct
MVLQTQVFISLLLWISGAYGEIVLTQSPDFQSVTPKEKVTITCSASSSASYMHWYQQKPDQSPKRWIYDTSKLASGVPSRFSGSGSGTDYTLTINSLEAEDAATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 232 aa, synthetic construct
MVLQTQVFISLLLWISGAYGEIVLTQSPATLSLSPGERATLSCSASSSASYIHWYQQKPGQAPRRWMYDTSKLASGIPARFSGSGSGTDYTLTISSLEPEDAAVYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 38 - protein, 233 aa, synthetic construct; identical to SEQ ID NO: 4